(19) United States
(12) Patent Application Publication
(10) Pub. No.: US 2008/0077150 A1
(43) Pub. Date: Mar. 27, 2008

(54) STEERABLE RASP/TRIAL MEMBER INSERTER AND METHOD OF USE

(76) Inventor: Linh Nguyen, Randolph, MA (US)

Correspondence Address: CARR LLP (IST), 670 FOUNDERS SQUARE, 900 JACKSON STREET, DALLAS, TX, 75202, US

(21) Appl. No.: 11/860,480

(22) Filed: Sep. 24, 2007

Related U.S. Application Data
(60) Provisional application No. 60/826,716, filed on Sep. 22, 2006.

(51) Int. Cl. A61B 17/00; A61B 17/58

(52) U.S. Cl. 606/85; 606/99

(57) ABSTRACT

A surgical instrument for posterior or lateral placement of a rasp/trial member between adjacent vertebrae comprising a first vertebrae and a second vertebrae may have an elongated member having a proximal end portion and a distal end portion and an articulation member slidingly coupled to the elongated member. A link member having a distal end portion and a proximal end portion may be pivotably coupled at the proximal end portion of the link member to a distal end portion of the articulation member. A rasp/trial member, pivotably coupled to the distal end portion of the elongated member may be pivotably coupled to the distal end portion of the link member. The rasp/trial member may include at least one surface for traumatizing tissue. An actuating mechanism coupled to the proximal end portions of elongated member and articulation member may be configured to move the articulation member relative to the elongated member.

### CROSS REFERENCE TO RELATED APPLICATIONS

This application relates to, and claims the benefit of the filing date of: co-pending U.S. provisional patent application Ser. No. 60/826,716 entitled “Steerable Rasp/Trial Inserter and Method of Use” filed Sep. 22, 2006 the entire contents of which are incorporated herein by reference for all purposes. This application also relates to: U.S. provisional patent application Ser. No. 60/825,091 entitled “Steerable Rasp/Trial Inserter” filed on Sep. 8, 2006; and U.S. provisional patent application Ser. No. 60/825,084 entitled “Instruments for Delivering Spinal Implants” filed on Sep. 8, 2006; U.S. provisional patent application Ser. No. 60/752,544 entitled “Reticulated Delivery Instrument” filed on Dec. 21, 2005; and U.S. provisional patent application Ser. No. 60/785,318 entitled “Spinal Implant Delivery Instrument” filed on Mar. 23, 2006, and U.S. patent application docket no. 06-010-US3 entitled “Removable Rasp/Trial Member Insert, Kit and Method of Use” filed concurrently herewith, the entire contents of which are hereby incorporated by reference.

### TECHNICAL FIELD

The invention relates generally to instruments and methods for spinal surgery and, more particularly, to steerable instruments used for preparing, inserting, and positioning, interbody devices or spacers in the intervertebral space of a human spine.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (e.g., bending either forward/anterior or aft/posterior), roll (e.g., lateral bending to either the left or the right side) and rotation (e.g., twisting of the shoulders relative to the pelvis).

The intervertebral spacing (i.e., between neighboring vertebrae) in a healthy spine is maintained by a compressible and somewhat elastic disc. The disc functions to allow the spine to move about the various axes of rotation and through the various arcs and movements required for normal mobility. The elasticity of the disc maintains the spacing between the vertebrae, allowing room or clearance for the compression of neighboring vertebrae, such as during the flexion and lateral bending of the spine. In addition, the elasticity of the disc allows relative rotation of neighboring vertebrae about a vertical axis, thereby allowing the twisting of the shoulders relative to the hips and pelvis. Clearance between neighboring vertebrae maintained by a healthy disc is also important to allow nerves from the spinal cord to extend out of the spine, between neighboring vertebrae, without being squeezed or impinged by the adjacent vertebrae.

In situations (e.g., based upon injury or otherwise) where a disc is not functioning properly, the inter-vertebral disc tends to compress, and in doing so pressure is exerted on the nerves extending from the spinal cord by this resulting reduced intervertebral spacing. As a result, various other types of nerve problems may be experienced in the spine, such as exiting nerve root compression in neural foramen, passing nerve root compression, and enervated annulus (i.e., where nerves grow into a cracked/compromised annulus, causing pain every time the disc/annulus is compressed), as examples.

Many medical procedures have been devised to reduce or alleviate such nerve compression and the pain that typically results from pressure being applied to the nerves. Many of these procedures revolve around attempts to prevent the vertebrae from moving too close to each other by surgically removing an improperly functioning disc and replacing it with a lumbar interbody fusion (LIF) device or spacer. Although prior interbody devices, including LIF cage devices, can be effective at improving a patient's overall condition, the vertebrae of the spine, body organs, the spinal cord, other nerves, and other adjacent body structures make it difficult to obtain surgical access to the locations between the vertebrae in which the LIF cage is to be installed.

Generally speaking, the surface or ends of the vertebrae adjacent to the spacer need to be decorticated prior to inserting the spacer into the intervertebral space. The decortication leaves the end surfaces of the vertebrae hemorrhaging, thereby promoting bone growth from the vertebrae. Subsequently, the growing bone envelopes the spacer and fuses the adjacent vertebrae together. However, the geometry of the vertebrae and surrounding tissue makes it difficult to insert decortication instruments into the intervertebral space. For similar reasons, moving or manipulating the decortication instruments (e.g., to clean or remove the boney material) is also difficult. What is needed, therefore, are instruments for decorticating vertebrae in a minimally invasive manner.

Prior to inserting a verterbal implant, a surgeon may want to insert a trial implant/instrument to determine the appropriate size of the implant to use. Various trial implants/instruments may be inserted and removed from the disc space before the surgeon is able to determine the proper size for the vertebral implant. However, the geometry of the vertebrae and surrounding tissue makes it difficult to insert trial instruments into the intervertebral space. For similar reasons, moving the trial implant instruments in order to position the various trial implants in their proper locations is also difficult. What is needed, therefore, are instruments that are configured to insert trial implants between adjacent vertebrae in a minimally invasive manner.

These and other features, and advantages, will be more clearly understood from the following detailed description taken in conjunction with the accompanying drawings. It is important to note that the drawings represent one illustrative embodiment from among many, and are not intended to represent the only aspect of the invention.

### SUMMARY

In an embodiment of the present invention a surgical instrument for posterior or lateral placement of a rasp between adjacent vertebrae comprising a first vertebrae and a second vertebrae is provided. The surgical instrument may have an elongated member having a proximal end portion and a distal end portion and an articulation member slidingly coupled to the elongated member. The articulation member may have a distal end portion and a proximal end portion. A link member having a distal end portion and a proximal end portion may be pivotably coupled at the proximal end portion of the link member to the distal end portion of the articulation member. A rasp member, pivotably coupled to the distal end portion of the elongated member may be pivotably coupled to the distal end portion of the link member. The rasp member may include at least one surface for traumatizing tissue, and may be interchangeable with a trial insert member for determining the space between adjacent bony structures. The surgical instrument may also have an actuating mechanism coupled to the proximal end portions of elongated member and articulation member configured to move the articulation member relative to the elongated member.

### BRIEF DESCRIPTION OF THE DRAWINGS

For a more complete understanding, reference is now made to the following descriptions taken in conjunction with the accompanying drawings, in which:

FIG. 1 illustrates a side view of an exemplary embodiment of a steerable rasp/trial instrument;

FIG. 2 illustrates an exploded view of the instrument illustrated in FIG. 1;

FIG. 3 illustrates an oblique view of one possible embodiment of a rasp/trial member that may be incorporated into an instrument such as the instrument illustrated in FIG. 1;

FIG. 4 illustrates an oblique view of one possible embodiment of a link that may be incorporated into an instrument such as the instrument illustrated in FIG. 1;

FIG. 5 illustrates an oblique view of one possible embodiment of a rasp/trial insert that may be incorporated into an instrument such as the instrument illustrated in FIG. 1;

FIG. 6 illustrates an oblique view of one possible embodiment of a rasp/trial insert and one possible embodiment of a mating distal end portion of an insertion instrument prior to being assembled;

FIG. 7 illustrates a side view of the distal end portion of the instrument illustrated in FIG. 1;

FIG. 8 illustrates a top view of one possible embodiment of a elongated member that may be incorporated in the instrument illustrated in FIG. 1;

FIG. 9 illustrates an oblique view of the proximal end portion of the elongated member shown in FIG. 8;

FIG. 10 illustrates a side view of one possible embodiment of an articulation member that may be incorporated in the instrument of FIG. 1;

FIG. 11 illustrates a top view of the proximal end portion of the articulation member shown in FIG. 10;

FIG. 12 illustrates a bottom view of the proximal end portion of the instrument member shown in FIG. 10;

FIG. 13 illustrates a top view of a proximal end portion of one possible embodiment of an elongated member mated to an articulation member that may be incorporated into the instrument illustrated in FIG. 1;

FIG. 14 is a cross-sectional view of a knob that may be incorporated into the instrument illustrated in FIG. 1;

FIG. 15A illustrates a cross-sectional view of one possible embodiment of an actuator mechanism in a first position that may be incorporated into the instrument illustrated in FIG. 1;

FIG. 15B illustrates an enlarged vie of a distal end portion of FIG. 1 in a first position;

FIG. 16A illustrates a cross-sectional view of one possible embodiment of an actuator mechanism in a second position that may be incorporated into the instrument illustrated in FIG. 1;

FIG. 16B illustrates an enlarged view of a distal end portion of FIG. 1 in a second position;

FIG. 17A is a top view of one possible embodiment of the instrument illustrated in FIG. 1 being inserted between two vertebral bodies;

FIGS. 17B and 17C respectively illustrate one possible embodiment of a distal end portion of the instrument illustrated in FIG. 1 in a first and a second position;

FIG. 18A is an cross-sectional side view of second possible embodiment of a steerable rasp/trial instrument;

FIG. 18B is an exploded view of the instrument illustrated in FIG. 18A;

FIG. 18C is a cross-sectional side view detail of one possible embodiment of an actuator mechanism shown in a first position and one possible embodiment of a locking member shown in a first position which may be incorporated into the instrument illustrated in FIG. 18A;

FIG. 18D is a cross-sectional side view detail of one possible embodiment of an actuator mechanism shown in a second position and one possible embodiment of a locking member shown in a first position which may be incorporated into the instrument illustrated in FIG. 18A;

FIG. 18E is a cross-sectional side view detail of one possible embodiment of an actuator mechanism shown in a third position and one possible embodiment of a locking member shown in a second position which may be incorporated into the instrument illustrated in FIG. 18A;

FIG. 18F is a cross-sectional side view detail of the instrument illustrated in FIG. 18A showing a rasp/trial member in a first position;

FIG. 18G is a cross-sectional side view detail of the instrument of FIG. 18A showing a rasp/trial member in a second position;

FIG. 18 H is a cross-sectional side view detail of the instrument of FIG. 18A showing a rasp/trial member in a third position;

FIG. 19A is a perspective view of a third possible embodiment of a steerable rasp/trial instrument;

FIG. 19B is a cross-sectional side view of the instrument illustrated in FIG. 19A;

FIG. 19C is a detail view of the area indicated within circle 19C of FIG. 19B;

FIG. 19D is an exploded perspective view of the area indicated within circle 19C of FIG. 19B;

FIG. 19E is a detail perspective view of one possible embodiment of a rasp/trial and linkage which may be incorporated in the instrument illustrated in FIG. 19A;

FIG. 19F is a detail top view of a distal end portion of an elongated member of the instrument illustrated in FIG. 19A; and

FIG. 20 illustrates one possible embodiment of a rasp/trial member instrument kit.

### DETAILED DESCRIPTION

The entire contents of the following provisional patent applications are incorporated herein by reference for all purposes: U.S. provisional patent application Ser. No. 60/826,716 entitled “Steerable Rasp/Trial Inserter and Method of Use” filed Sep. 22, 2006; U.S. provisional patent application Ser. No. 60/825,091 entitled “Steerable Rasp/Trial Inserter” filed on Sep. 8, 2006; U.S. provisional patent application Ser. No. 60/825,084 entitled “Instruments for Delivering Spinal Implants” filed on Sep. 8, 2006; Ser. No. 60/752,544 entitled “Reticulated Delivery Instrument” filed on Dec. 21, 2006; and U.S. provisional patent application Ser. No. 60/785,318 entitled “Spinal Implant Delivery Instrument” filed on Mar. 23, 2006.

For the purposes of promoting an understanding of the principles of the present inventions, reference will now be made to the illustrative embodiments, or examples, shown in the drawings. Specific language will be used to describe the same. It will nevertheless be understood that no limitation of the scope of the invention is thereby intended with regard to the drawings or to the language used in the Specification. Any alterations and further modifications in the described embodiments, and any further applications of the principles of the inventions as described herein are contemplated as would normally occur to one skilled in the art to which the invention relates.

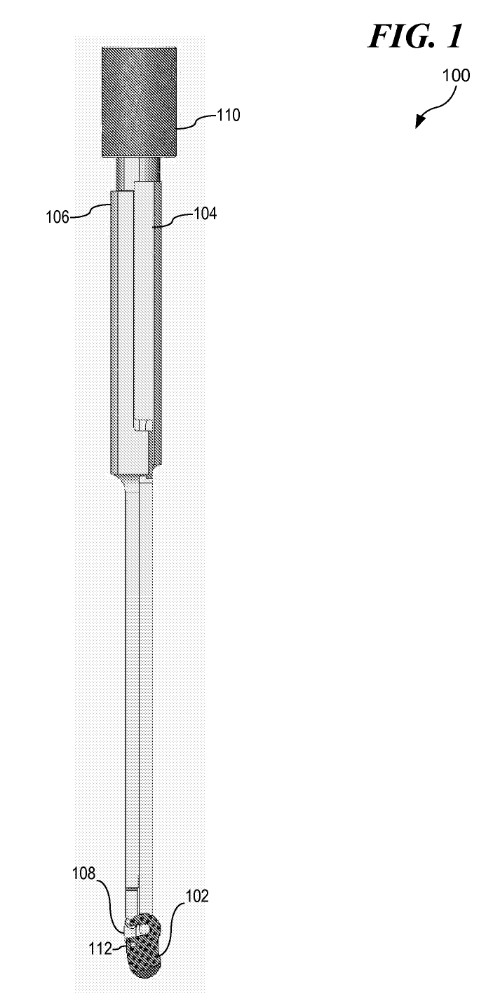

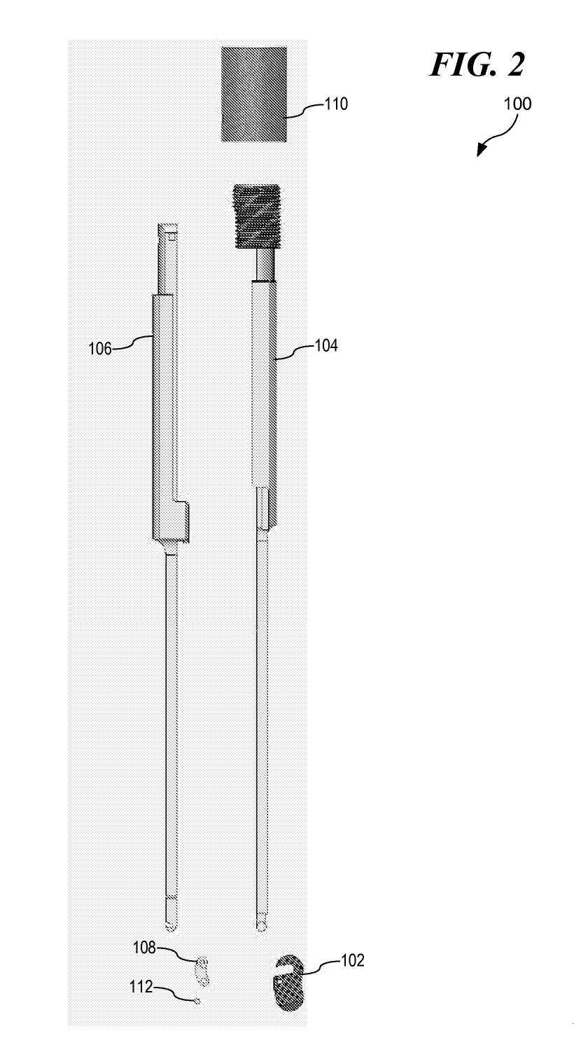

Turning now to FIGS. 1 and 2, one possible embodiment of a steerable rasp/trial member instrument 100 is presented in the drawings. The instrument 100 may include a rasp/trial member 102, an elongated member 104, an articulation member 106, a link 108, a handle 110, and pin 112. As will be discussed in greater detail below, the instrument 100 may be used to scrape away the surface of an adjacent vertebra so as to prepare the vertebra for a fusion procedure in which a vertebral implant may be used. As will be discussed in greater detail below, the steerable rasp/trial member instrument 100 may also be used to insert a trial member in-between two adjacent vertebrae in order to determine the appropriate sized vertebral implant to insert. Accordingly, the rasp/trial member 102, the elongated member 104, the articulation member 106, the link 108, the knob 110, and the pin 112, may interact to provide the proper angular motion and transmit the force required to access the intervertebral space between two adjacent vertebrae.

Referring to FIG. 2, an exploded view is shown illustrating one possible embodiment of a steerable rasp/trial member instrument 100. In some embodiments, elongated member 104 and articulation member 106 may be coupled or mated with knob 110 so as to form an articulation mechanism. By rotating the knob 110, the articulation member 106 may move or translate relative to the elongated member 104. This action may drive the angular pivoting of the steerable rasp/trial member 102. In certain embodiments, rasp/trial member 102 may be coupled to connector 108 using pin 112 and configured such that the components form a pivoting mechanism.

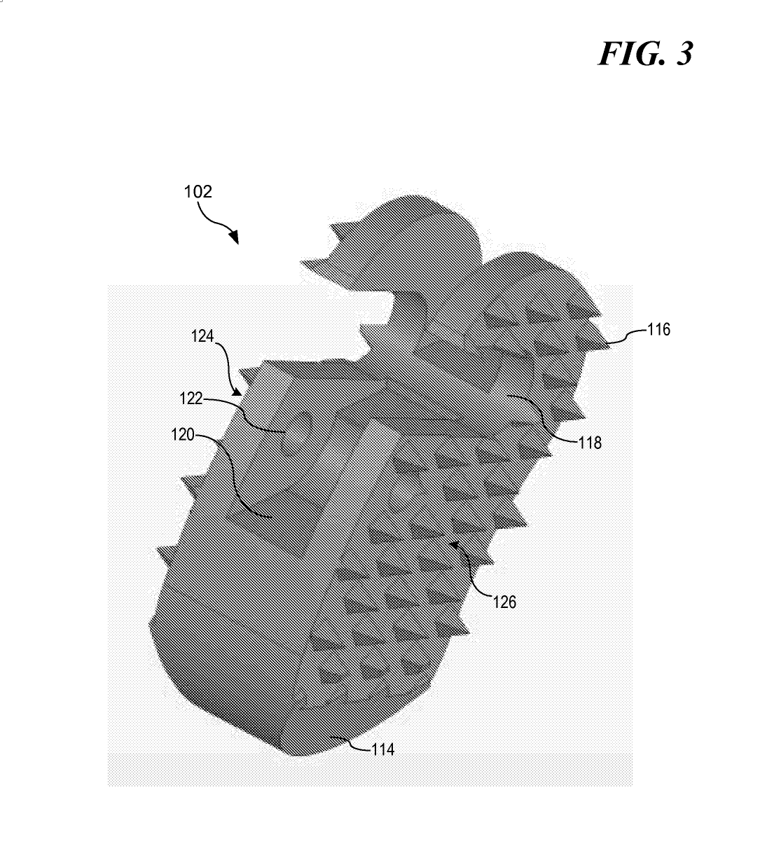

Referring now to FIG. 3, this drawing illustrates a detailed enlarged orthogonal view of one embodiment of the rasp/trial member 102. The rasp/trial member 102 may be generally kidney shaped and may have a chamfer or lead-in 114 at its distal end to aid in insertion of rasp/trial member 102 between two adjacent vertebrae (not shown). The rasp/trial member 102 may include a top surface 126 and a bottom surface 124. The chamfer or lead-in 114 may be on one surface of the rasp/trial member 102 or on more than one surface (chamfer or lead-ins 114 are shown on the first and second surfaces 124, 126 in this example). The angular landing shown for the chamfer or lead-in 114 is for illustration only. Some embodiments may be configured in a spheroidal or arcuate shape (not shown) in order to aid in the insertion of the rasp/trial member 102.

The top and/or bottom surfaces 126, 124 of rasp/trial member 102 may have a plurality of projections 116 to aid in scraping or decorticating bone from the adjacent vertebrae. FIG. 3 shows the plurality of projections 116 configured substantially as spikes, but the projections 116 may also be configured as teeth, ridges, or other appropriate shapes of various heights and angles. Other embodiments may comprise a rough or textured surface. In certain embodiments, such as a trial member implant, it may be advantageous to have the top and bottom surfaces 126, 124 of rasp/trial member 102 substantially smooth so that the rasp/trial member instrument 100 may be easily inserted and removed.

In certain embodiments the rasp/trial member 102 may have side surfaces that connect the top and bottom surfaces 126, 124. Rasp/trial member 102 may also have an instrument slot 118 located near a proximal end. The instrument slot 118 may extend transversely from one side surface of the rasp/trial member 102 towards an opposing side surface. Instrument slot 118 may be useful for mating or coupling a rasp/trial member 102 to a rasp/trial member instrument 100 (FIGS. 1 and 2). In certain embodiments, slot 118 may be generally u-shaped. In some embodiments rasp/trial member 102 may have connector slot 120 that extends partially through one of the side surfaces. The connector slot 120 may aid in connecting the rasp/trial inserter 102 to a distal end of a rasp/trial member instrument 100 or connecting link 108 (FIGS. 1 and 2). Rasp/trial member 102 may include a bore 122 that extends through the top surface 126, connector slot 120, and bottom surface 124. Bore 122 may aid in facilitating the pivoting and connecting of the rasp/trial member 102 to a rasp/trial member instrument 100 or a connecting link 108 (see FIGS. 1 and 2).

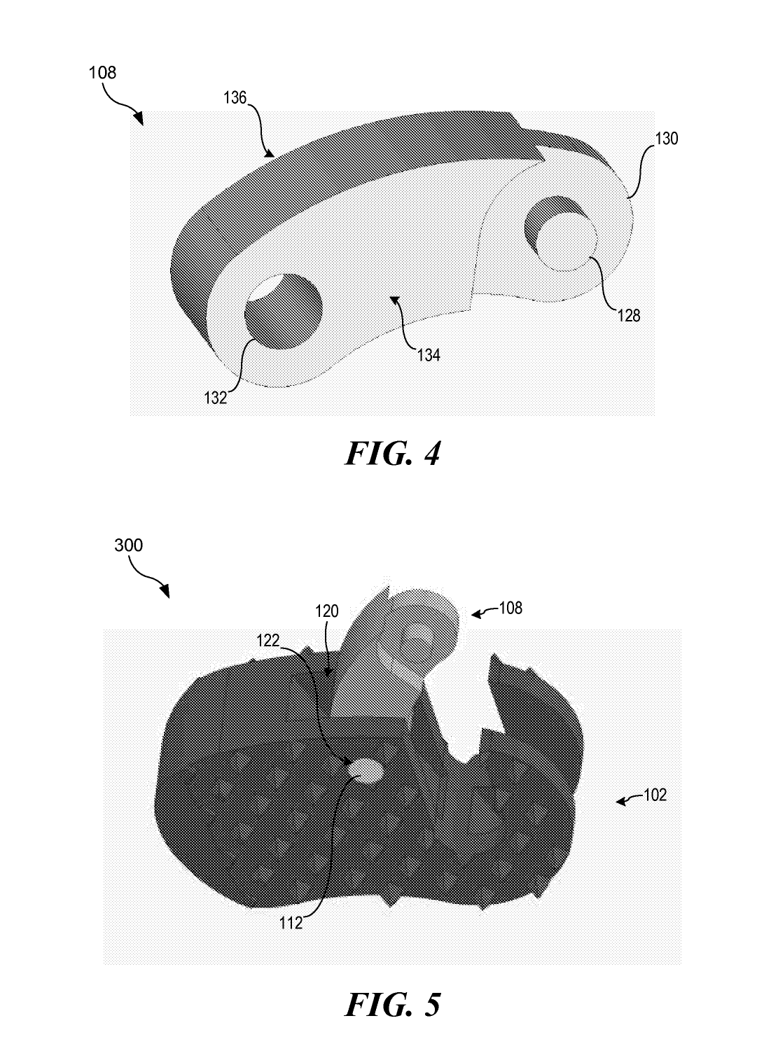

Turning now to FIG. 4, reference number 108 indicates a connector link shown in an orthogonal view. The link 108 may comprise a top surface 134 and a bottom surface 136. In other embodiments, the link 108 may comprise more than one link member (not shown). In some embodiments link 108 may have arcuate side walls that extend along a curved longitudinal axis and connect the top and bottom surfaces 134, 136. Link 108 may further have arcuate proximal and distal walls that are shorter than the side walls. In some illustrative embodiments, link 108 may have a proximal end portion 130 that may be recessed from the top and/or bottom surfaces 134, 136 and/or the side and proximal walls. As shown in FIG. 4, the proximal end portion 130 is recessed from the top and bottom surfaces 134, 136.

Furthermore, in the present example, the proximal end portion of link 108 may have one or more projections 128 that extend from the recessed top and/or bottom surfaces 134, 136 (only one may be seen in this view, but two projections 128 are present in this embodiment). Projection 128 may be configured in the shape of a pin or a tab and may be integral with the link 108 or separate and coupled to the link 108. The substantially cylindrical pin shape shown in FIG. 4 for projection 128 is for the purposes of illustration only. The projection 128 may be any of a variety of configurations suitable for the purpose of coupling to the distal end portion of a rasp/trial member instrument 100 (FIGS. 1 and 2). In certain embodiments, the distal end portion of link 108 may comprise an orifice or bore 132 that extends through the top and bottom surfaces 134, 136.

In other embodiments, the projection 128 and the bore 132 may be respectively located in the distal and proximal end portions of the link 108. Additionally, the link 108 may comprise a first and second projection 128, or a first and second bore 132 in place of the configuration shown in the embodiment in FIG. 4. In such situations, the corresponding connecting portions of the rasp/trial member 102 and rasp/trial member instrument 100 would configured accordingly (see FIGS. 1 and 2).

Referring now to FIG. 5, the distal portion of link 108 may be inserted into connector slot 120 as shown in the illustrative embodiment. A pin 112 may then be inserted through the rasp/trial member bore 122 (more clearly shown in FIG. 3) and the connector bore 132 (as shown in FIG. 4) to pivotally couple the link 108 to the rasp/trial member 102. The resulting rasp/trial member insert assembly 300 may be used for rasp/trial members 102 of various sizes and geometries. The rasp/trial member insert assembly 300 may readily facilitate quick and simple interchangeability of various rasp/trial member insert assemblies 300 and the rasp/trial member instrument 100. As will be described in greater detail below, various kits may be used that contain rasp/trial members 102 and/or rasp/trial member insert assemblies 300 of different sizes and geometries.

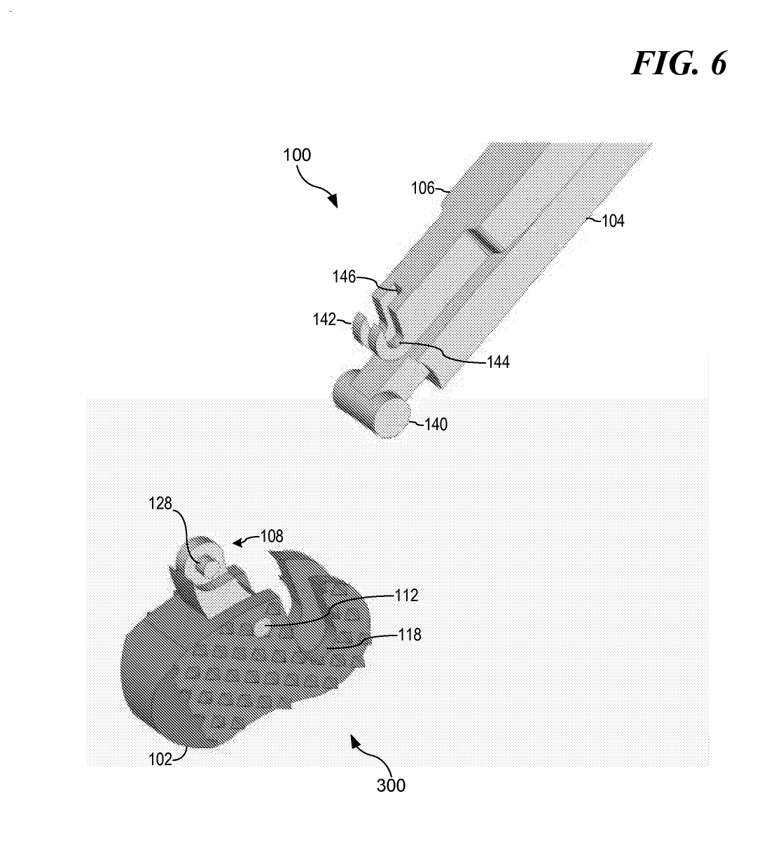

Now referring to FIG. 6, an illustrative embodiment of a distal portion of a steerable rasp/trial member instrument 100 is shown prior to being connected to a rasp/trial member insert assembly 300. The rasp/trial member instrument 100 may comprise an elongated member 104. The elongated member 104 may be configured to have a shaped end portion 140 to which the rasp/trial member insert assembly 300 may be pivotally coupled. The shaped end portion 140 is shown in a T-bar configuration, but other geometries and configurations may be used that are able to capture rasp/trial member 102 while also allowing the captured rasp/trial member 102 to pivot relative to the elongated member 104. For example, one skilled in the art should appreciate that an L-shaped end portion, among others, may be used in place of a T-bar shape. Shaped end portion 140 may be dimensioned or configured to be received within the instrument slot 118 of the rasp/trial member 102.

Articulation member 106 may also have a shaped end portion 142 that couples with rasp/trial member insert assembly 300 to actuate pivotal motion of the rasp/trial member insert assembly 300 relative to shaped end portion 140 of elongated member 104 of rasp/trial member instrument 100. The shaped end portion 142 may have a hook shaped configuration as shown in FIG. 6. The shaped end portion 142 may have a U-shaped slot 144 that may be dimensioned to receive the projections 128 of the link 108 of the rasp/trial member insert assembly 300. Shaped end portion 142 may further comprise a longitudinal slot 146 that may extend partially into the distal end portion of the shaped end portion 142. The longitudinal slot 146 may be dimensioned or configured to accommodate connector 108 of rasp/trial member insert assembly 300.

The rasp/trial member insert assembly 300 and the distal end portion of the rasp/trial member instrument 100 may be configured to be coupled and un-coupled without any additional fasteners and/or actions required by the technician. The use of the pin and slot type assembly allows for a quick and simple coupling method to rapidly attach and detach a rasp/trial member insert assembly 300 from the end of a rasp/trial member instrument 100. The specific placement of the pins versus the slots is for the purposes of illustration only. A person of skill in the art would recognize that the pin and slot arrangements (for example, other configurations may be used) may be switched around such that the link 108 comprises a slot and the articulation member 106 comprises a pin. In some embodiments, the link 108 may be pivotally coupled to the distal end portion of the articulation member 106 via a pin and detachably coupled to the rasp/trial member 102 via a pin and slot arrangement. Other variations may be within the knowledge of a person of skill in the art.

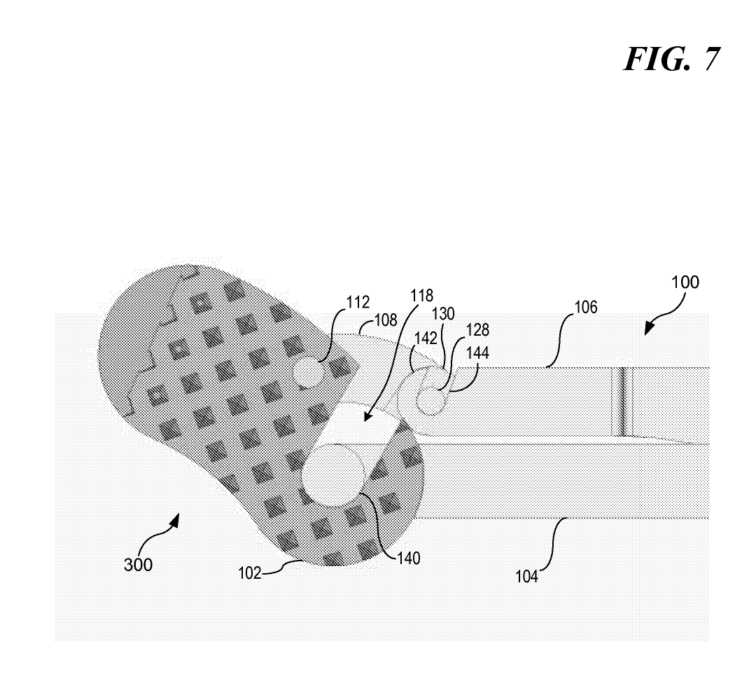

Assembly of the rasp/trial member insert assembly 300 to an embodiment of the rasp/trial member instrument 100 is illustrated in FIG. 7. Shaped end portion 140 of the elongated member 104 may slide into slot 118 located in the rasp/trial member 102. The projections 128 of link 108 may slide into slot 144 of shaped end portion 142. The rasp/trial member insert assembly 300 may be coupled to the distal end portion of the rasp/trial member instrument 100 through rotation and transverse sliding movement. For example, recessed proximal end portion 130 of link 108 may slide into the longitudinal slot 146 (not visible in this view, refer to FIG. 6) of the distal end of the articulation member 106. Rasp/trial member 102 may then rotate about pin 112 and/or projections 128 to aid in positioning shaped end member 140 within slot 118 of the rasp/trial member 102. After assembly, the rasp/trial member 102 may be configured to pivot or rotate about shaped end 140 of the elongated member 104.

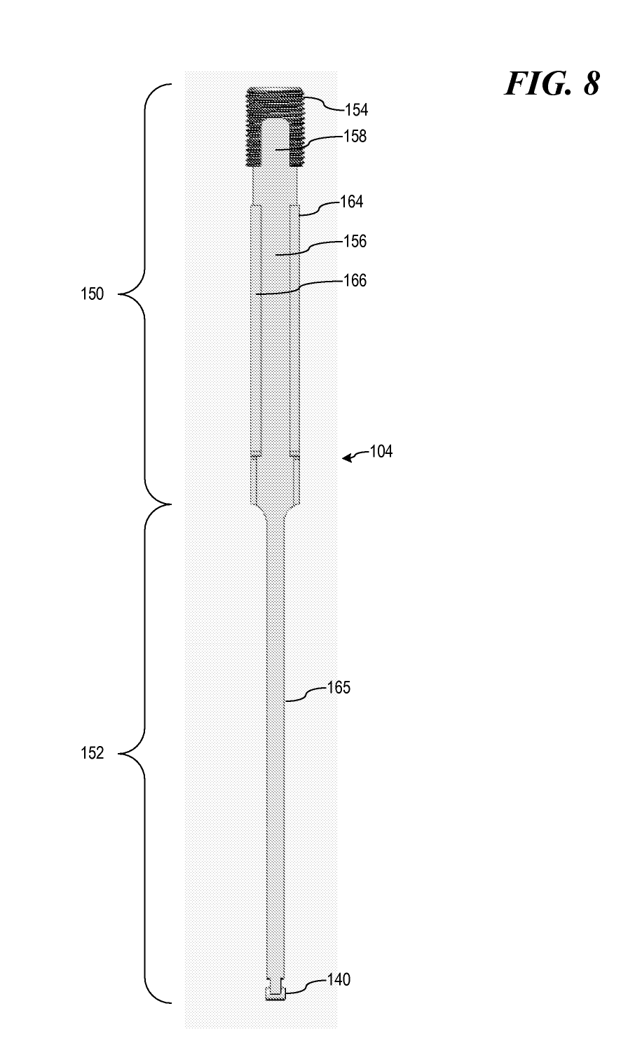

Turning now to FIG. 8, this drawing shows an illustrative embodiment of the elongated member 104. The elongated member 104 may comprise a handle portion 150 and a shaft portion 152. The handle portion 150 may comprise a generally cylindrical threaded proximal end portion 154 and an abutment surface 156. Abutment surface 156 may extend partially into the threaded proximal end portion 154, forming a slot 158 in a top face of the cylindrical threaded end portion 154. Abutment surface 156 may have side walls 164 and 166 that are axially spaced apart from threaded proximal end portion 154 and terminate prior to shaft portion 152. In some embodiments, the side walls 164 and 166 may function to retain the articulation member 106 as shown in FIG. 1. Shaft portion 152 may further comprise a shaft 165 that extends longitudinally from the handle portion 150. The distal end portion of shaft 165 may comprise a shaped end 140 (as discussed earlier).

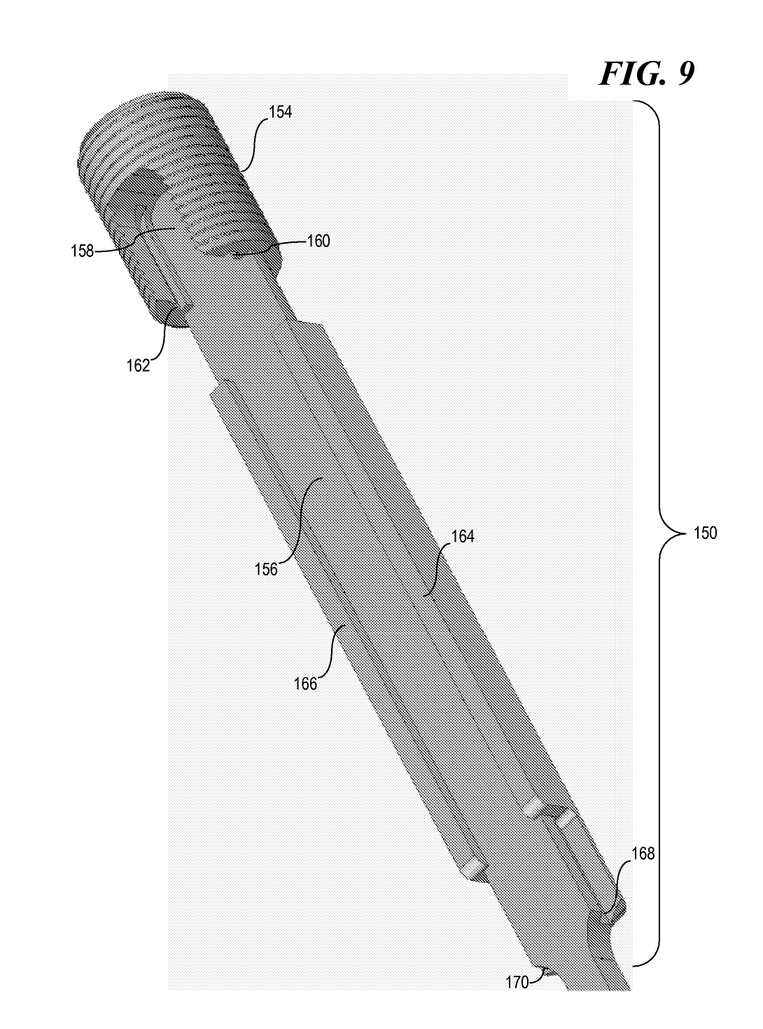

Referring now to FIG. 9, the cylindrical threaded end portion 154 may contain one or more grooves 160 and 162 located on one or both of the side walls of slot 158. Grooves 160 and 162 may extend from the distal end portion of threaded proximal end portion 154 along its longitudinal axis and may terminate at the back face of slot 158. The distal end portion of the handle portion 150 may comprise grooves 168 and 170 that extend longitudinally along the side walls of the distal end portion of handle portion 150. Grooves 168 and 170 may begin at the distal end of handle portion 150 and may terminate at side walls 164 and 166 of the abutment surface 156. The grooves 160, 162, 168, and 170, may be configured to prevent or inhibit the proximal and mid-sections of the articulation member 106 from separating from the elongated member 104 (FIGS. 1 and 2).

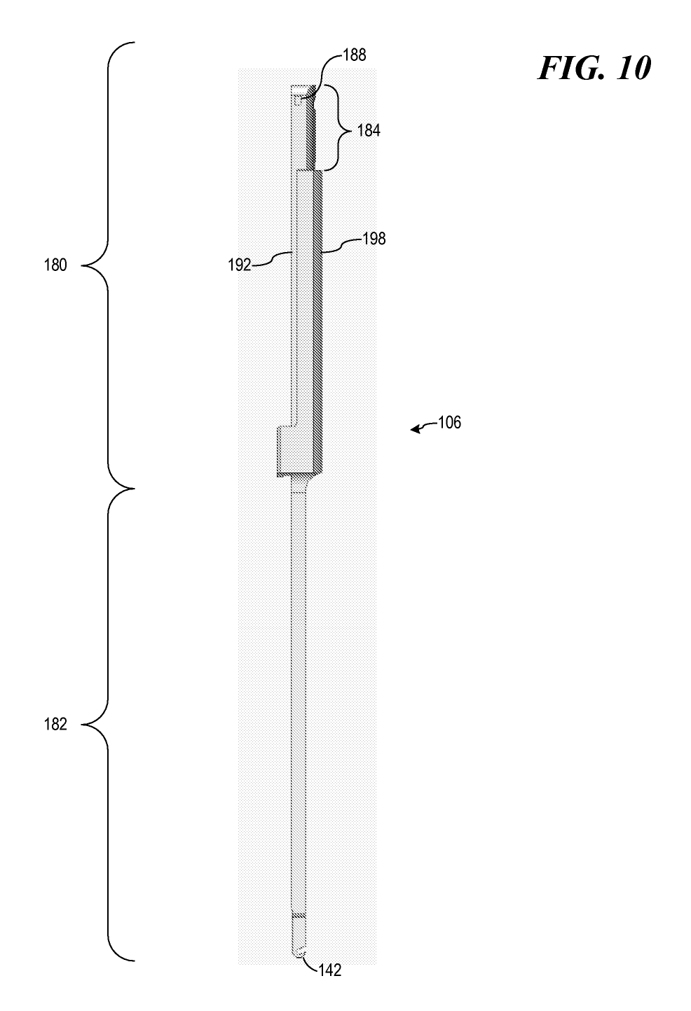

Turning now to FIG. 10, this drawing shows a side view of an illustrative embodiment of the articulation member 106. The articulation member 106 may comprise a handle section 180 and a shaft section 182. The proximal end section 184 of the handle section 180 may be configured to couple with the slot 158 of the articulation member 106 (FIGS. 8 and 9). The handle section 180 may further comprise a bottom surface 192 and a top surface 198. The bottom surface 192 may abut the abutment surface 156 of the elongated member 104 (FIGS. 8 and 9). The handle section 180 may comprise a proximal recessed section 184 further comprising one or more tabs 188 (only one is visible in this view). The surface of the proximal recessed section 184 may be offset from the surface of the adjoining top surface 198, as shown in the example illustrated in FIG. 10. The shaft section 182 may extend in a longitudinal direction from the handle section 180. The shaft section 182 may comprise the shaped end portion 142 (as discussed earlier).

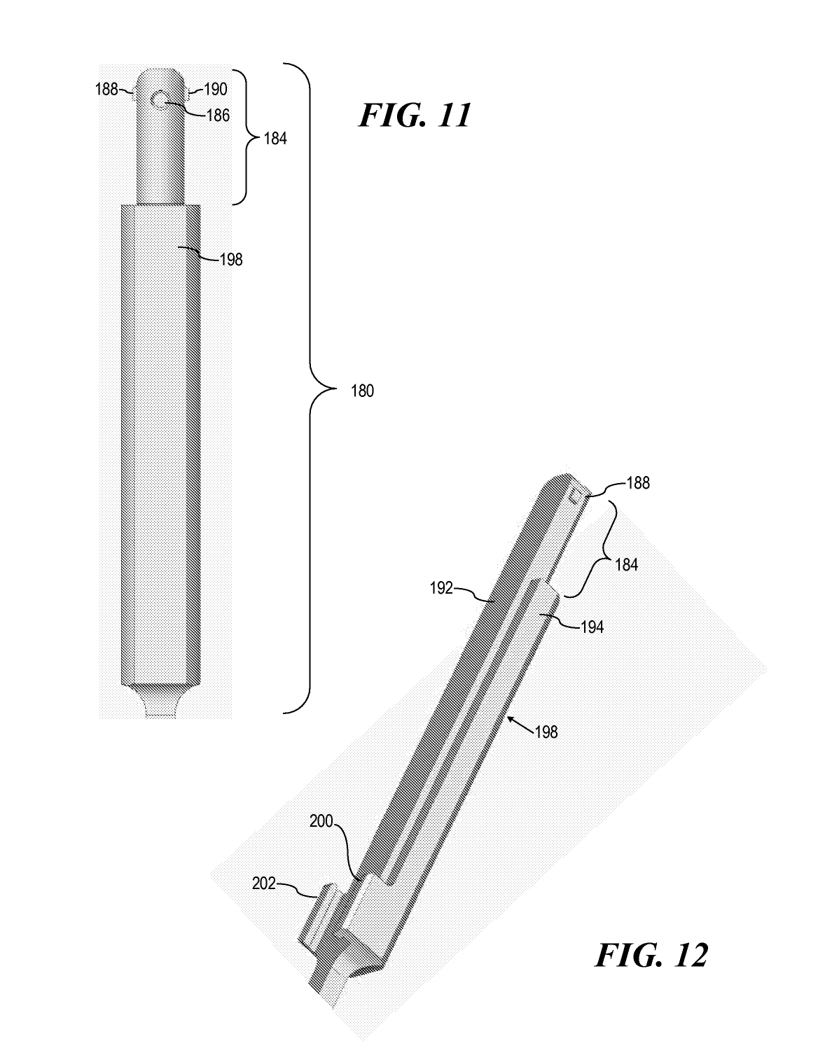

Referring now to FIG. 11, this drawing shows an enlarged top view of the handle section 180 of FIG. 10. In this view, the offset of the surface of the proximal recessed section 184 is shown with respect to the side walls and top surface 198 of the handle section 180. Additionally, tabs 188 and 190 are visible on either side of the proximal recessed section 184. Although tabs 188 and 190 are shown as substantially rectangular in this exemplary embodiment, the invention may not be limited to this geometry or configuration for these components. Any number of tabs in a variety of shapes, lengths, and configurations may be used for the tabs 188, 190 provided on the proximal recessed section 184. The tabs 188 and 190 may be integral to the proximal recessed section 184 and/or they may be separately coupled to the proximal recessed section 184 (e.g., such as pins or rods, among others). The proximal recessed section 184 may further comprise an orifice or bore 186. The bore 186 may be located between the tabs 188 and 190 for example, or any convenient location along the proximal recessed section 184. The bore 186 may be used to secure a pin (220, not shown) as will be described later in greater detail.

Turning now to FIG. 12 an enlarged view of handle section 180 is illustrated. The proximal recessed section 184 of handle section 180 may have a bottom surface 192, two side walls 194 (only one side wall is visible in this view) and top surface 198. Bottom surface 192 may abut the abutment surface 156 of elongated member 104 (FIGS. 8 and 9). In some embodiments, the bottom surface 192 may be configured to slide along the abutment surface 156 of the elongated member 104. In other embodiments, the two surfaces may engage each other through an intermediate member, such as a roller bearing for example. Bottom surface 192 is shown as a flat surface, but the invention may not be limited to this embodiment. Bottom surface 192 may be concave or convex to correspond with the geometry and configuration of the abutment surface 156.

Side walls 194 may begin at the distal end of proximal recessed section 184 and may extend longitudinally to the shaft section 182. Side walls 194 may be recessed or offset from the bottom surface 192 and extend to the top surface 198. The distal end portion of side walls 194 may have substantially L-shaped projections 200, 202. The L-shaped projections 200, 202 may extend below and partially apart from the bottom surface 192. The L-shaped projections 200, 202 may be configured to receive grooves 168, 170 of articulation member 106 (FIG. 9).

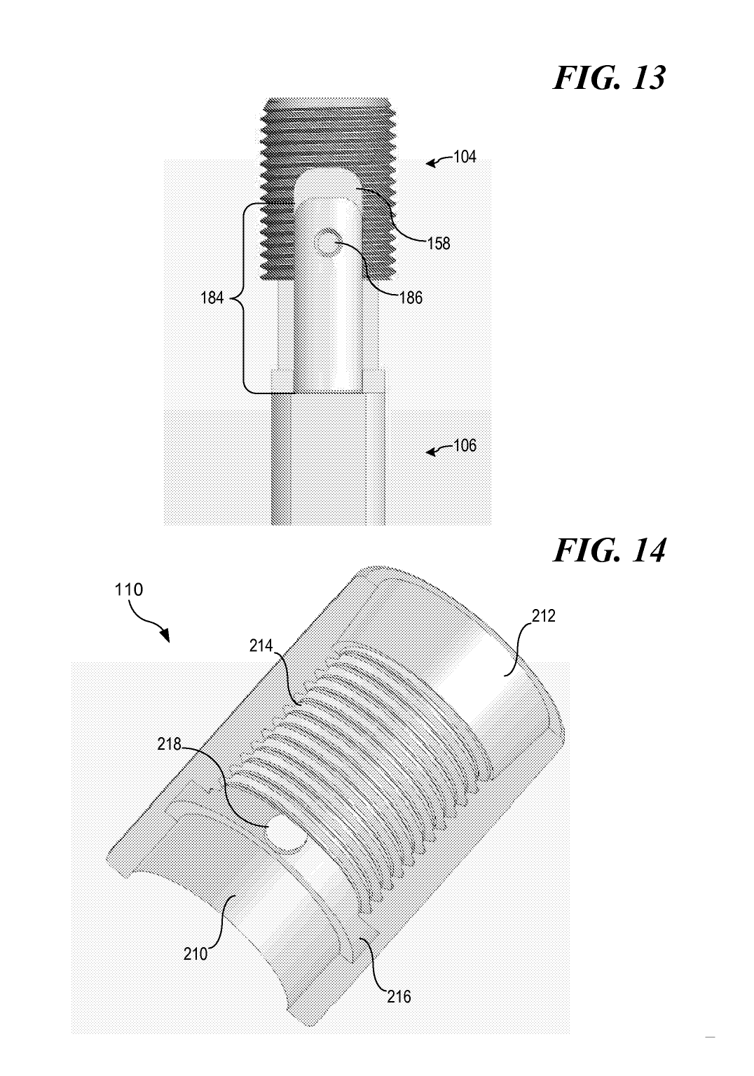

Referring now to FIG. 13, this drawing shows a proximal end portion of the articulation member 106 assembled to a proximal end portion of the elongated member 104. As shown in this illustrative embodiment, the proximal recessed section 184 of the articulation member 106 may be dimensioned to slidingly couple with the slot 158 of elongated member 104. Due to the engagement of the tabs 188, 190 (FIG. 11), with the grooves 160, 162 (FIG. 9), the articulation member 106 may translate relative to the elongated member 104. However, the articulation member 106 may be restrained from separating from the elongated member 104 in a direction perpendicular to the translating movement (e.g., out of the plane containing the drawing). In certain embodiments, the bore 186 is located substantially along the middle in the transverse direction of the proximal recessed section 184.

Referring now to FIG. 14, this drawing shows a cross-sectional view of one possible embodiment of the knob 110. The knob 110 may have a generally cylindrical shape with a bore extending there through. The internal face of knob 110 may have a relatively smooth bore 210 and 212 (respectively) at proximal and distal end portions, and a threaded section 214 interposed between the two. In addition, the knob 110 may comprise a radial groove 216 located between the smooth distal bore 210 and the threaded section 214. The groove 216 may further comprise a bore 218 that establishes a communication pathway between the interior of the knob 110 and the exterior. The bore 218 may provide a pass through for a pin described later.

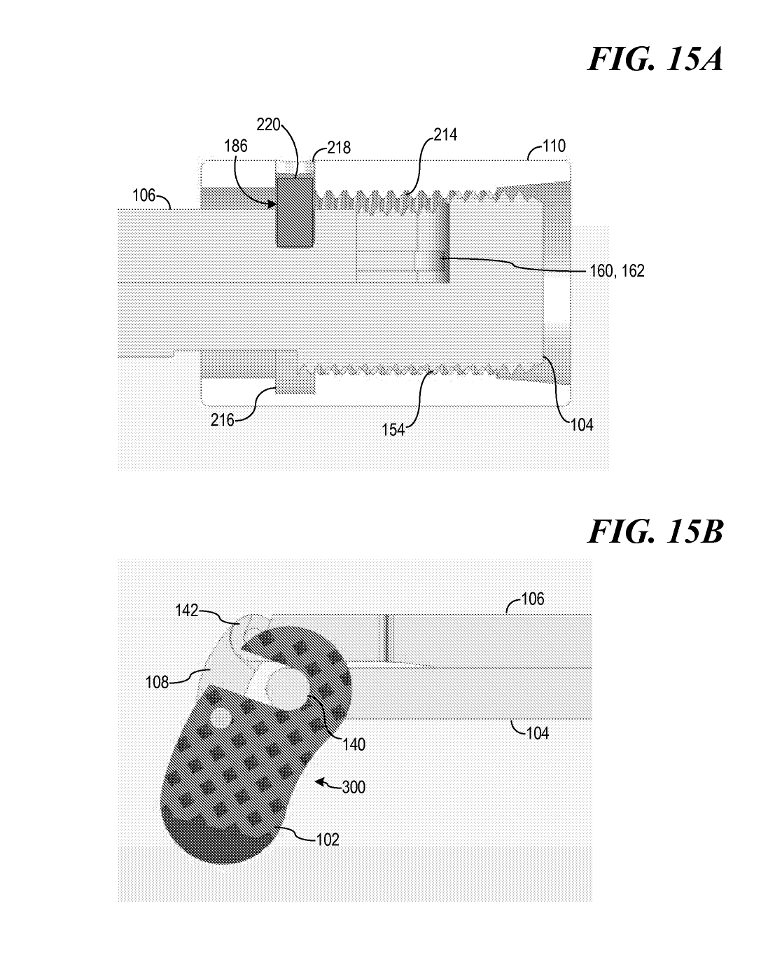

Referring now to FIGS. 15A and 15B, these drawings show an one possible embodiment of an actuator mechanism in a first position. FIG. 15A illustrates a cross-sectional view of a first position of the actuator mechanism that may result from relative positions of the knob 110, the elongated member 104, and the articulation member 106. FIG. 15B illustrates a first position which may result from a first position of the rasp/trial member insert assembly 300.

In addition, the threaded portion 214 of the knob 110 may be threadably coupled to the cylindrical threaded end portion 154 of elongated member 104. In such a situation, bore 218 of knob 110 may line up with bore 186 of articulation member 106. A pin 220 may be passed through bore 218 and coupled to the bore 186, with at least a portion of the pin 220 extending above the surrounding surface of the articulation member 106. The pin 220 may then engage the radial groove 216, fixing the position of the articulation member 106 relative to the knob 110 and the elongated member 104. Rotating the knob 110 may slide the pin 220 along the radial groove 216, maintaining the position of the knob 110 relative to the articulation member 106. However, rotating the knob 110 may move the knob 110 along the cylindrical threaded end portion 154 of the elongated member 104, changing the position of the knob 110 relative to the elongated member 104.

As shown in FIG. 15A, the knob 110 is at a distal location relative to the elongated member 104. Accordingly, the articulation member 106 may be at a distal location as well. The grooves 160, 162 are visible in the cross-section near the proximal end of the elongated member 104. As stated earlier, this may be considered a first position for the actuation mechanism comprising the knob 110, articulation member 106, elongated member 104 and pin 220.

As shown in FIG. 15B, the first position of the actuation mechanism may comprise the shaped end 142 extending longitudinally beyond the shaped end 140. This may be due to articulation member 106 being positioned at a distal location relative to the elongated member 104. Accordingly, shaped end 142 of the articulation member 106 may apply a force to the link member 108, pivoting the rasp/trial member 102 about the shaped end 140. This position may represent a first limit to the pivoting of the rasp/trial member insert assembly 300

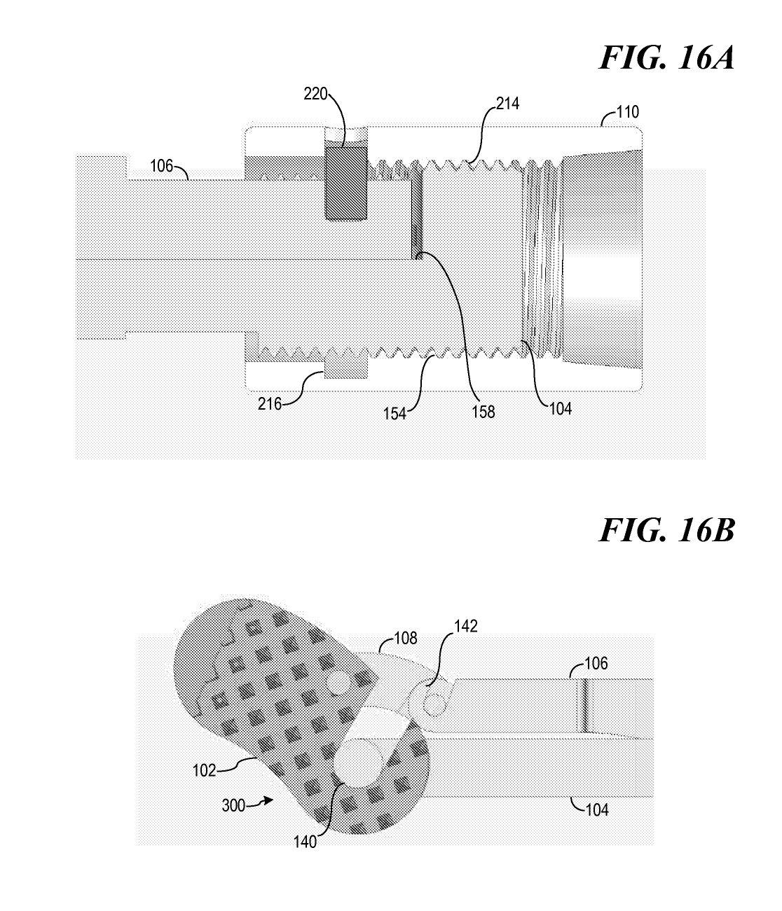

Turning now to FIGS. 16A and 16B, these drawings show one possible embodiment of an actuator mechanism in a second position. FIG. 16A illustrates a cross-sectional view of a second position of the actuator mechanism that may result from relative positions of the knob 110, the articulation member 106, and the elongated member 104. FIG. 16B illustrates the results of the second position relative to the rasp/trial member insert assembly 300. As shown in FIG. 16A, one possible embodiment of an articulation member 106 may be coupled to elongated member 104 and configured to translate relative to one another.

As shown in FIG. 16A, the knob 110 is located at the proximal end portion of the elongated member 104. Accordingly, articulation member 106, fixed in a longitudinal direction with respect to the knob 110 by the pin 220, may also be at a proximal limit with respect to the elongated member 104. As seen in the cross-section, the articulation member 106 may be near the limit of translational movement defined by the slot 158. As detailed above, the knob 110 may translate in an axial direction relative to the elongated member 104 due to an interaction between the threaded portion 214 of the knob 110 and the cylindrical threaded end portion 154. The movement of the knob 110 may correspondingly move the articulation member 106 to a proximal position, otherwise known as the second position.

As seen in FIG. 16A, when articulation member 106 moves axially to a second position, the shaped end portion 142 may translate proximally relative to shaped end portion 140 of the elongated member 104. The shaped end portion 140 may exert a force in the distal direction at a proximal end of the rasp/trial member insert assembly 300 while the shaped end portion 142 may exert a force in the proximal direction at a proximal end portion of the link 108. The interaction of the shaped ends 140, 142, may cause the rasp/trial member insert assembly 300 to pivot about shaped end portion 140. The pivoting motion of the rasp/trial member insert assembly 300 may be controlled along an arc by link 108. The orientation shown in FIG. 16B substantially represents a possible second limit to the motion of the rasp/trial member 102 of the rasp/trial member insert assembly 300 relative to the articulation member 106 and the elongated member 104. In certain embodiments rasp/trial member 102 may have up to 180 degrees of angulation or rotational motion, preferably between −30 degrees to +90 degrees relative to the elongated member 104. Any number of positional angular relationships may be possible between the elongation member 104 and the rasp/trial member 102 and are not limited to the extents described above.

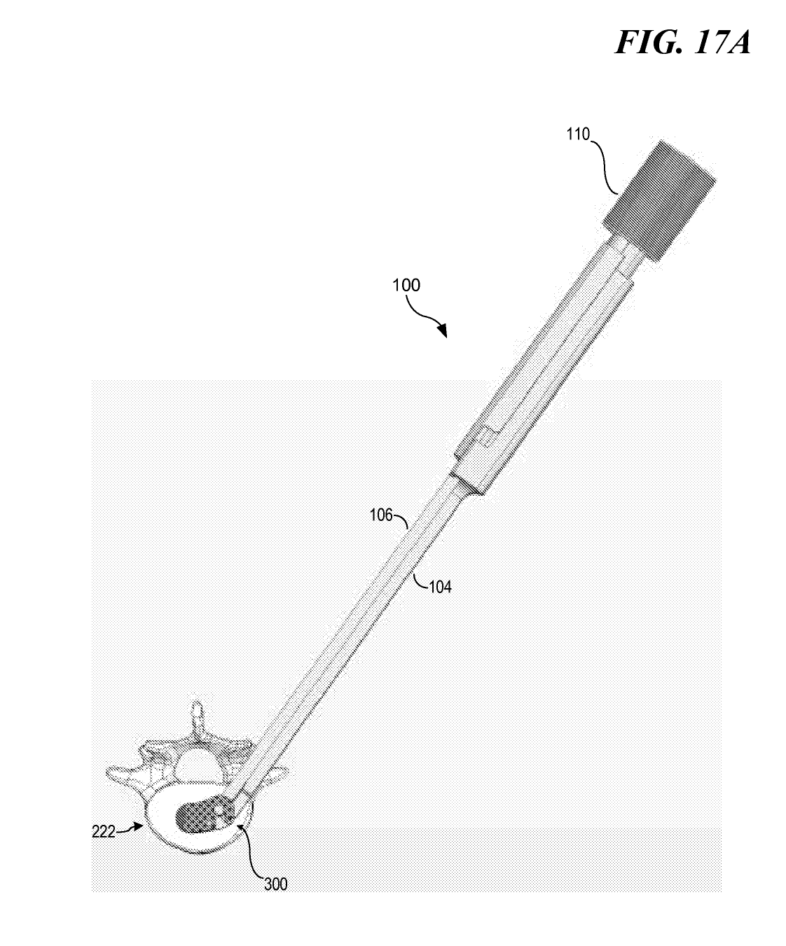

One possible embodiment of a manner or method for using an instrument such as the instrument 100 is illustrated in FIGS. 17A, 17B, and 17C. The rasp/trial member insert assembly 300 shown in FIG. 7 may be coupled to the distal end portions of the elongated member 104 and the articulation member 106 as described above. The distal end portion of instrument 100 may then be inserted into the intervertebral space between two vertebral bodies 222. The instrument 100 may use a posterior lateral (shown in FIG. 17A), a posterior medial, or a direct posterior approach, among others.

Once inserted between two vertebral bodies 222, the knob 110 may be rotated to a first position (see FIG. 15A). The surgeon may then move the instrument 100 back and forth in order to scrape the adjacent surfaces of the two vertebral bodies 222. The surgeon may further rotate the knob 110 to pivot the rasp/trial member insert assembly 300 into a second position (see FIG. 16A). The surgeon may move the instrument 100 back and forth again. This process may be repeated several times and the instrument 100 may be pivoted through an almost infinite combination of positions between and including the first position and the second position, in order to remove the desired amount of bone from surfaces of the vertebral bodies 222. If a surgeon desires to remove bone either more or less quickly from the vertebral bodies 222, the surgeon may replace rasp/trial member insert assembly 300 (FIGS. 15B, 16B) with a rasp/trial member insert assembly 300 comprising a rasp member of a different size or shape (as will be described in greater detail below).

The instrument 100 may also be used to determine the correct or appropriate size of implant to use in the intervertebral space. In such a situation, the surgeon may or may not initially decorticate the boney surfaces of the vertebral bodies 222 in order to remove a layer of bone and begin the hemorrhaging of the surfaces. Either way, the distal end portion of the instrument 100 may be inserted into the intervertebral space between two vertebral bodies 222 (FIG. 17A). The rasp/trial member insert assembly 300 may be pivoted and pushed until located in the position desired by the surgeon. At this point, the surgeon may then determine if the rasp/trial member insert assembly 300 is too large or too small for the intervertebral space. If the surgeon believes the current fit is inadequate or faulty, he/she may remove the instrument 100 from the intervertebral space between the vertebral bodies 222 and replace the rasp/trial member insert assembly 300 (FIG. 5) with a rasp/trial member insert assembly 300 of a more appropriate size. The surgeon may repeat the previous process of inserting and positioning the instrument 100 within the intervertebral space between the vertebral bodies 222 as many times as necessary to achieve a proper size determination for the intervertebral space.

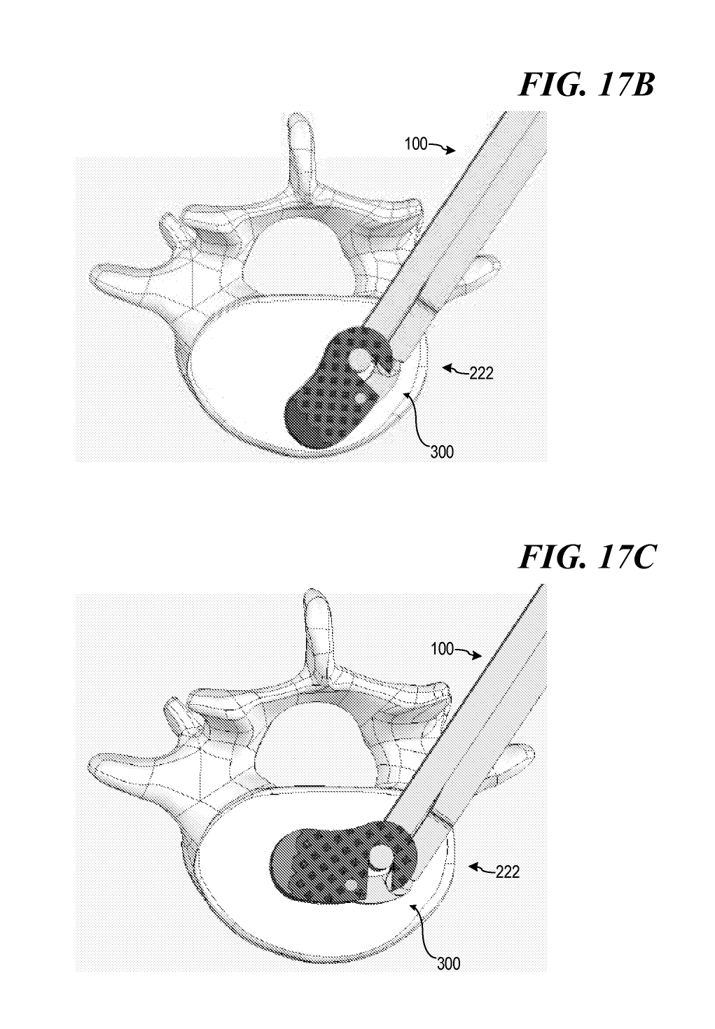

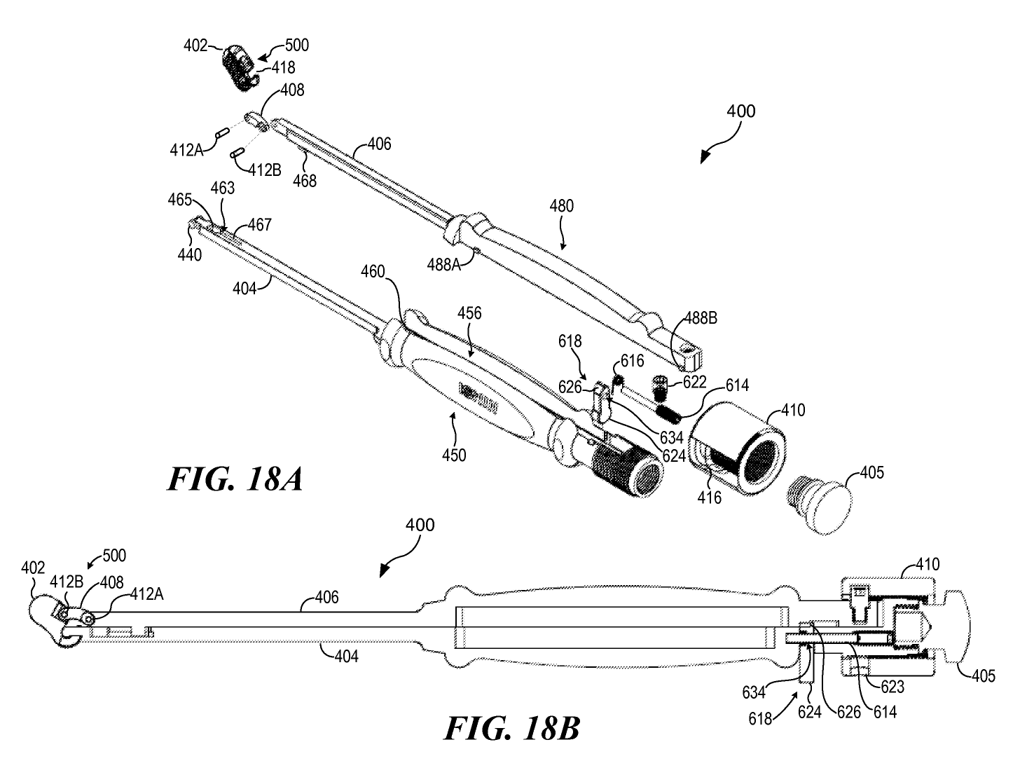

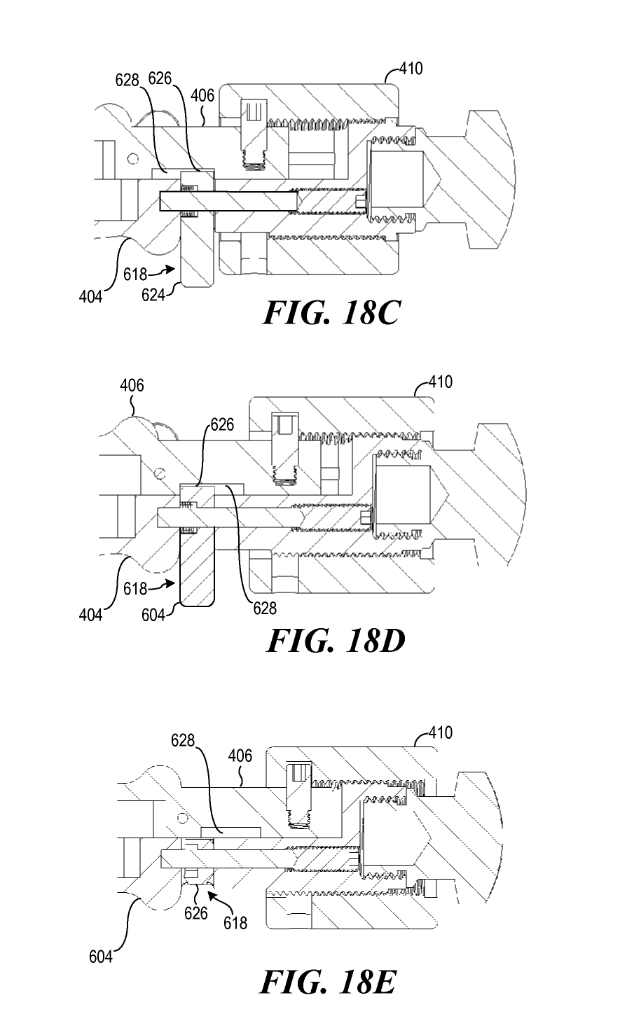

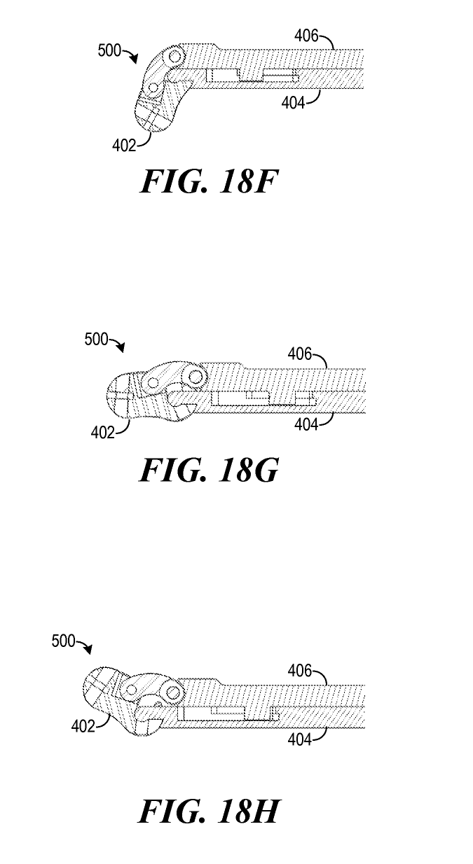

Turning now to FIGS. 18A and 18B, a second possible embodiment of a steerable rasp/trial instrument 400 is shown. The instrument 400 comprises features that may enable a rasp/trial member 402 to be pivotally attached to the instrument 400 generally as described hereinbefore, inserted into an intervertebral space, rotated therein for decorticating the adjacent vertebra or determining if rasp/trial member 402 is too large or small and withdrawn from the space. More particularly, the instrument 400 may comprise a knob 410, an elongated member 404, an articulation member 406, and an insert link 408. Additionally, the instrument 400 may comprise an impact head 405, lock pivot 14, and lock member 318. The components of the instrument 400 will be described in more detail below.

The articulation member 406 may be slidingly coupled to the elongated member 404. The articulation member 406 may be configured to translate relative the elongated member 404 without becoming separated. More particularly, a handle section 480 of articulation member 406 may fit within a channel 456 formed in a handle section 450 of elongated member 404. The handle section 480 of articulation member 406 may be captured within the channel 456 of handle section 450 by capture features 488, which may be pins or tabs or other devices protruding slightly from the handle section 480 of articulation member 406, which capture features 488 may fit slidingly within small channels or grooves 460 formed along lower corners of channel 456. Further, the articulation member 406 may include a tab 468 on an underside and near a distal end portion of the articulation member, which tab 468 may slidingly fit within a slot 463 formed in a top side and near a distal end portion of the elongated member 404. Tab 468 may be configured as a T-shaped member that may be inserted through a wide portion 465 of slot 463 and subsequently be captured by a narrow portion 467 of slot 463. Thus, the articulation member 406 may be slidingly coupled to the elongated member 404 by the use of tabs 468 and tab 488 slidingly coupled to grooves 463 and to slot 460, respectively.

Further, the articulation member 406 may be coupled to the knob 410 via a pin 412C (not shown). The pin 412C may slidingly interact with a radial groove 416 located internal to the knob 410. The knob 410 may be threadably engaged with a proximal end of the elongated member 404. Rotation of the knob 410 may translate the location of the pin 412C relative to the elongated member 404. The amount of translation may be related to the longitudinal distance traveled by the knob 410 as the knob 410 travels along the threaded section of the end of the elongated member 404. Translation of the pin 412C relative to the elongated member 404 may result in a corresponding translation of the articulation member 406 relative to the elongated member 404.

The rasp/trial member insert assembly 500 may be connected to the instrument 400 at two separate locations. The rasp/trial insert assembly 500 may include a link member 408 pivotably coupled to the rasp/trial member 402 via pins 412A. The rasp/trial member 402 may be connected to the distal end portion of the elongated member 404 and configured so as to be able to pivot relative the elongated member 404 generally as described hereinbefore. The connection of the rasp/trial member 402 to the elongated member 404 may be through a pin connection for example or any of a variety of pivotable connections between two members known to a person of skill in the art. The connection may be through one side of the rasp/trial member 402 or two sides of the rasp/trial member 402. The rasp/trial member 402 may be recessed to prevent the side of the elongated member 404 from extending beyond the surface of the rasp/trial member 402. In certain embodiments, rasp/trial member 402 may be coupled via a slot 418 that may hook on to a T-shaped end portion 440 formed at a distal end portion of the elongated member 404, which may also serve as the pivot point for the rasp/trial member 402. In certain embodiments, the geometry of the pivotal connection may provide that rasp/trial member 402 be engaged or disengaged from the T-shaped end portion 440 at certain extreme angles of pivotal motion about a pivot point, and yet may not be disengaged at other angles throughout the range of pivotal motion.

The link member 408 of the rasp/trial member insert assembly 500 may also be pivotably connected to the articulation member 406 via a pin 412B. The link member 408 may be configured in an arcuate shape as shown, but the link member 408 is not to be limited to this particular shape. The link member 408 may be pivotally connected to both the articulation member 406 and the rasp/trial member 402. Therefore, and as described hereinbefore, translational movement of articulation member 406 with respect to elongated member 404 may cause rasp/trial member 402 as linked to articulation member 406 via link member 408 to pivot about a pivot point configured as shaped end portion 440 at the distal end portion of the elongated member 404.

The impact head 405 may be threadably attached to the proximal end portion of the handle section 450. The impact head 405 may allow an impact force to be transferred through the instrument 400 without applying an excessive force to either the articulation knob 410 or the threaded interface between the articulation knob 410 and the a handle section 450. As shown in FIG. 18A, the impact head 405 may be a separate piece secured to the proximal end portion of the a handle section 450. In some embodiments, the proximal end portion of the handle section 450 may extend beyond the articulation knob 410 and perform a function similar to the impact head 405.

The instrument 400 may comprise a releasable lock mechanism 618. The lock mechanism 618 may comprise an actuator portion 624, a cam portion 626, and a pivot orifice 634. The pivot orifice 634 may pivotably couple the lock mechanism 618 to the elongated member 404. The lock mechanism 618 may be biased in a locked position by a resilient biasing member 616 surrounding a lock pivot 614. The resilient biasing member 616 is shown in FIG. 18A as a torsion coil spring, however, the resilient lock member 616 may not be limited to this configuration. A wide variety of resilient members and locations may be used in a similar manner to the resilient lock member 616 and the lock pivot 614.

The lock member 618 may be coupled to the elongated member 404 via the lock pivot 614, and configured to rotate about the lock pivot 614 relative to the elongated member 404. The lock pivot 614 may be configured substantially in the shape of an elongated cylinder, smooth on a distal end portion and threaded on a proximal end portion. The distal end portion of the lock pivot 614 may be inserted into the pivot orifice 634 and the proximal end portion may be fixed to the elongated member 404. The lock mechanism 618 may normally be biased in a first position and may limit translational motion of articulation member 406 between a first and second longitudinal position with respect to elongated member 404. In certain embodiments the first position as illustrated in FIG. 18A-18B may be a “down-and-locked” position in which lock member 618 may extend radially outward from a longitudinal centerline of the instrument 400. In certain embodiments, the lock mechanism 618 may be pivoted to a second position in which lock mechanism 618 may extend in a generally transverse direction to the instrument 400 to unlock the instrument 400 for the purpose of removing and/or interchanging the rasp/trial member assembly 500.

Turning now to FIG. 18C-18H, one possible embodiment of the lock mechanism 618 may be shown to interact with articulation member 406 and may releasably limit an extent of retraction, or translational motion in a proximal direction, with respect to elongated member 404. As shown in FIGS. 18C and 18F, lock mechanism 618 may normally be biased in a first position and rasp/trial member assembly 500 may be in a first angular position. In this first position the cam portion 626 of lock mechanism 618 extends into a cavity 628, such as a slot or recess, formed in an underside of articulation member 406. A proximal end portion of cavity 628 may abut a proximal side portion of cam portion 626 and may thereby limit translational movement of articulation member 406 in a distal direction with respect to elongated member 404, thereby limiting an extent to which articulation member 406 may extend with respect to elongated member 404.

As shown in FIGS. 18D and 18G, with locking mechanism 618 in the first position, the articulation member 406 may be placed in a second position and rasp/trial member assembly 500 may be moved to a second angular position. For example, the articulation member 406 may be translated in a proximal direction by rotational action of knob 410 as described above, until a distal end portion of cavity 628 abuts a distal side portion of cam portion 626 of lock mechanism 618, thereby limiting an extent to which articulation member 406 may retract with respect to elongated member 404. The limits of extension and retraction which may be governed by a length of cavity 628 may correspondingly limit a pivotal angle of travel of rasp/trial member assembly 500 with respect to the pivotal connection of rasp/trial member assembly 500 to the distal end portion of the elongated member 404.

As shown in FIGS. 18E and 18H, lock mechanism 618 may be pivoted to a second position in which cam portion 626 may be pivoted away from and out of cavity 628. With cam portion 626 effectively removed from cavity 628 such that no interior portion of cavity 628 abuts either proximal or distal side of cam portion 626, further rotational action of knob 410 may retract articulation member 406 further in a proximal translational direction, into a third position. The lock mechanism 618 may be restrained in the second position by an underside of articulation member 406 riding over and in contact with lock mechanism 618. Such further retraction, or proximal translational movement, may allow one limitation of pivotal angle of travel of the rasp/trial member assembly 500, which may correspondingly enable the rasp/trial member assembly 500 to become disengaged from its pivotal connection to the distal end portion of elongated member 404 and articulation member 406. Disengagement of the rasp/trial member assembly 500 from its pivotal connection to elongated member 404 and articulation member 406 enables the rasp/trial member assembly 500 to be unloaded from, exchanged, and/or reloaded to the instrument 400. Thereafter, rotational action of knob 410 may extend articulation member 406 in distal translational movement back to the second position illustrated in FIG. 18D, at which time the lock mechanism 618 may become freed from restraint by articulation member 406. Once freed from restraint by the articulation member 406, the resilient biasing member 616 (FIG. 18A) may positively urge the lock mechanism 618 into the first position (FIG. 18C or 18D), preferably with a characteristic and noticeable snap, so that the user may be assured that the rasp/trial member assembly 500 is positively attached to the instrument 400 within acceptable limits of pivotal angular movement and therefore may not become disengaged in vitro.

Turning now to FIG. 18F-18H, translational movement of the articulation member 406 may be shown to effect pivotal movement of the rasp/trial member assembly 500 and the rasp/trial member 402 about a pivot point. FIG. 18F depicts the rasp/trial member assembly 500 in a first angular position. The first angular position of the rasp/trial member assembly 500 may represent a first limit of pivotal motion that may also correspond to a first longitudinal position of the articulation member 406. The first longitudinal position of the articulation member 406 may represent a substantially fully extended position of articulation member 406, and may correspond to a position of the knob 410 as shown in FIG. 18C.

FIG. 18G depicts the rasp/trial member assembly 500 and the rasp/trial member 402 in a possible second angular position. The second angular position of the rasp/trial member assembly 500 may represent a second limit of pivotal motion that may correspond to a second longitudinal position of the articulation member 406 and may correspond to a position of the knob 410 as shown in FIG. 18D.

FIG. 18H depicts the rasp/trial member assembly 500 and the rasp/trial member 406 in third possible angular position. The third angular position may represent a third limit of pivotal motion, wherein rasp/trial member 402 may be disengaged from its pivotal coupling point. The third angular position of the rasp/trial member assembly 500 may correspond to a third longitudinal position of the articulation member 406. The third longitudinal position may represent a further retracted position of articulation member 406, and may correspond to a position of the knob 410 as shown in FIG. 18E. Therefore it may be seen that the rasp/trial member assembly 500 may pivot through a range of motion between a first limit and a second limit wherein the rasp/trial member assembly 500 may be securely coupled to its pivotal coupling point, and may also pivot to a further third limit where the rasp/trial member assembly 500 may be disengaged from its pivotal coupling point.

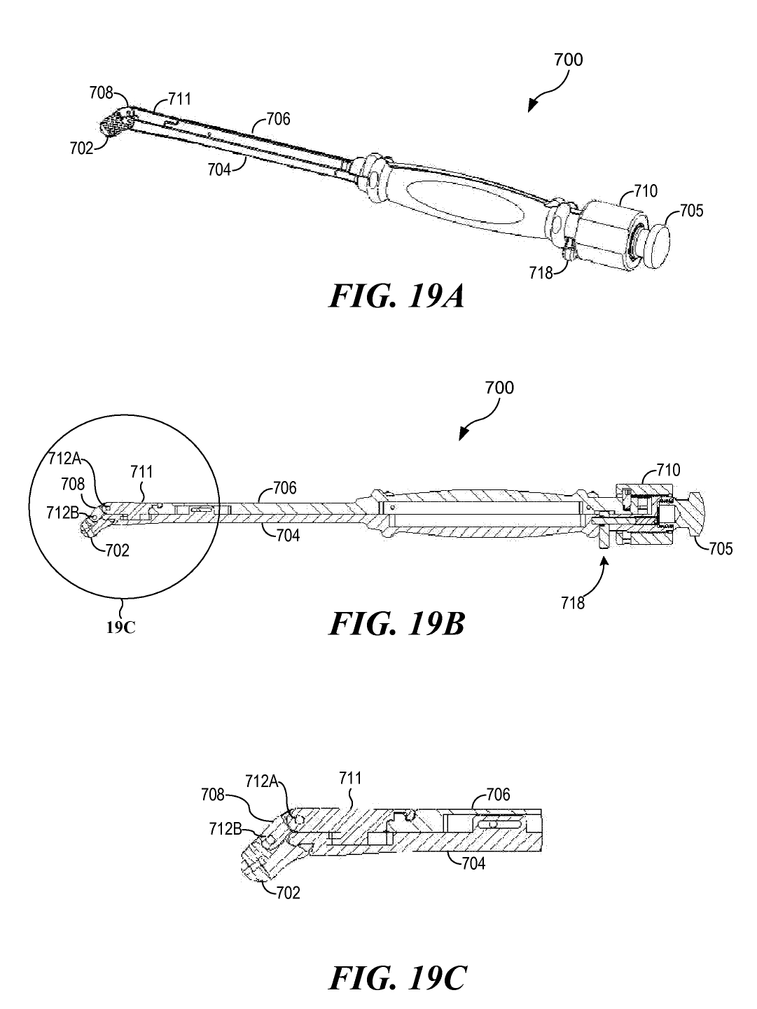

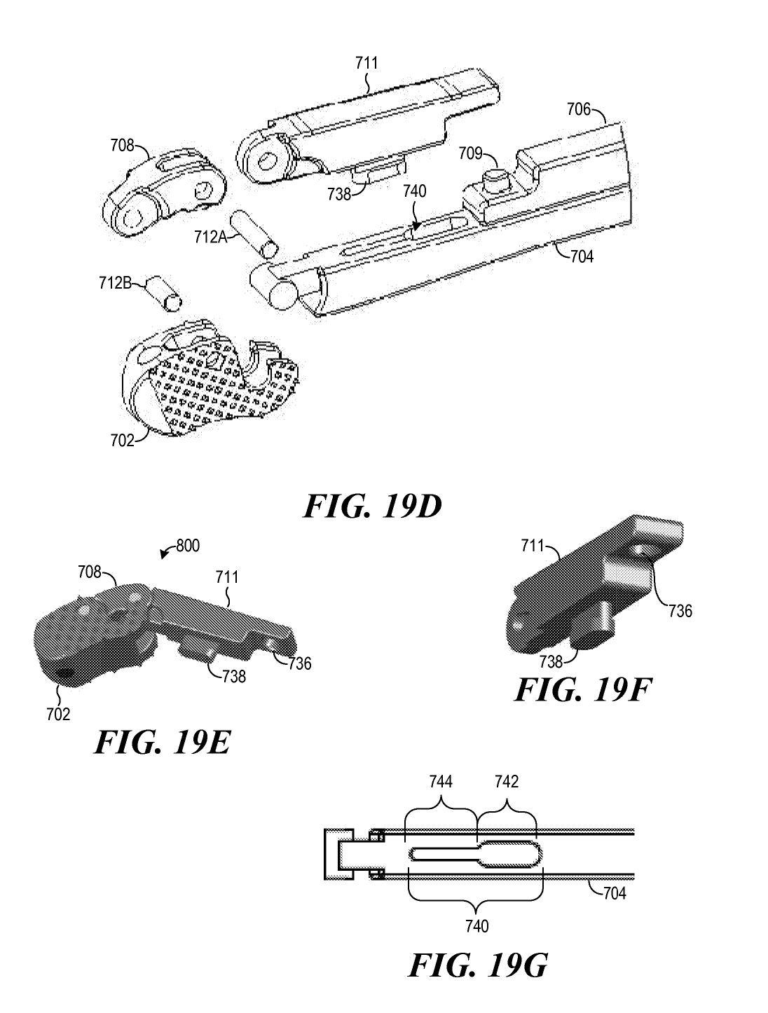

Turning now to FIG. 19A-C, a third possible embodiment of a steerable rasp/trial instrument 700 is shown. The instrument 700 may have similar features with similar functions as the previously described embodiments. For example the instrument 700 may have a locking mechanism 718, an impact head 705 and an actuator mechanism as described in FIGS. 18A-E. The instrument 700 may comprise features that enable a rasp/trial member 702 to be attached to the instrument 700, inserted into a vertebral space, rotated therein for decorticating (or for use as a trial) the adjacent vertebra, and withdrawn from the space. More particularly, the instrument 700 may comprise an articulation knob 710, an elongated member 704, an articulation member 706, a link member 708 and a translation member 711. The components of the instrument 700 will be described in more detail below.

The articulation member 706 may be slidingly coupled to the elongated member 704 generally as described hereinbefore. The articulation member 706 may be configured to translate relative the elongated member 704 without becoming separated. Further, the articulation member 706 may be coupled to the knob 710 via a pin 712. The pin 712 may slidingly interact with a radial groove located internal to the knob 710. The knob 710 may be threadably engaged with a proximal end portion of the elongated member 704. Rotation of the knob 710 may translate the location of the pin 712 relative to the elongated member 704. The amount of translation may be related to the longitudinal distance traveled by the knob 710 as the knob 710 travels along the threaded section of the elongated member 704. Translation of the pin 712 relative to the elongated member 704 may result in a corresponding translation of the articulation member 706 relative to the elongated member 704.

The rasp/trial member 702 may be connected to the instrument 700 at a distal end portion of the elongated member 704 and configured so as to be able to rotate relative to the elongated member 704. The connection of the rasp/trial member 702 to the elongated member 704 may be through pin connections, for example, or any of a variety of pivotable connections between two members known to a person of skill in the art. The connection may be through one side of the rasp/trial member 702 or two sides of the rasp/trial member 702. The rasp/trial member 702 may be recessed to prevent the side of the elongated member 704 from extending beyond the surface of the rasp/trial member 702.

Turning now to FIG. 19D-19G, the rasp/trial member 702 may be connected to the articulation member 706 via the link member 708 and the translation member 711. A rasp/trial member insert assembly 800, as shown in FIG. 19E, may include a rasp/trial member 702, the link member 708, the translation member 711 and pins 712. The link member 708 may be configured in an arcuate shape as shown, but the link member 708 may not to be limited to this particular shape. The link member 708 may be pivotably connected to both the rasp/trial member 702 and the translation member 711. The translation member 711 may be pivotally connected to the link member 708 and may further be coupled to the articulation member 706. A projection 709 formed in a distal portion of articulation member 706 may fit within a recess 736 formed in the translation member 711 to couple the translation member 711 to the articulation member 706. A second projection 738, such as a T-slot key, may be formed in the translation member 711 and may fit within a slot 740 forming in a distal portion of the elongated member 704 which may aid in slidingly coupling translation member 711 to the elongated member 704. The second projection 738 of translation member 711 may be aligned with the slot 740 in the elongated member 706 to install the translation member 711 onto the elongated member 704 through a wide portion 742 at a proximal end portion of the slot 740. As the instrument 700 is actuated to slide the articulation member 706 towards the distal end portion of the elongated member 704, the second projection 738 may slide along the slot 740 towards a narrow portion 744 at a distal end portion of the slot 740 thereby capturing translation member 711 the elongated member 704. The link member 708 may be pivotally coupled through the use of pins 712. For example, pins 712A, and 712B are shown in FIG. 19C-E as coupling the link member 708, via the translation member 711, to the instrument 700.

The handle/actuating mechanism illustrated in FIGS. 19A and 19B is similar to the handle/actuating mechanism illustrated in FIG. 18A-18E. For brevity and clarity, a description of those parts which are identical or similar to those described in connection with the embodiment illustrated in FIGS. 18A to 18E will not be repeated here. Reference should be made to the foregoing paragraphs with the following description to arrive at a complete understanding of this embodiment.

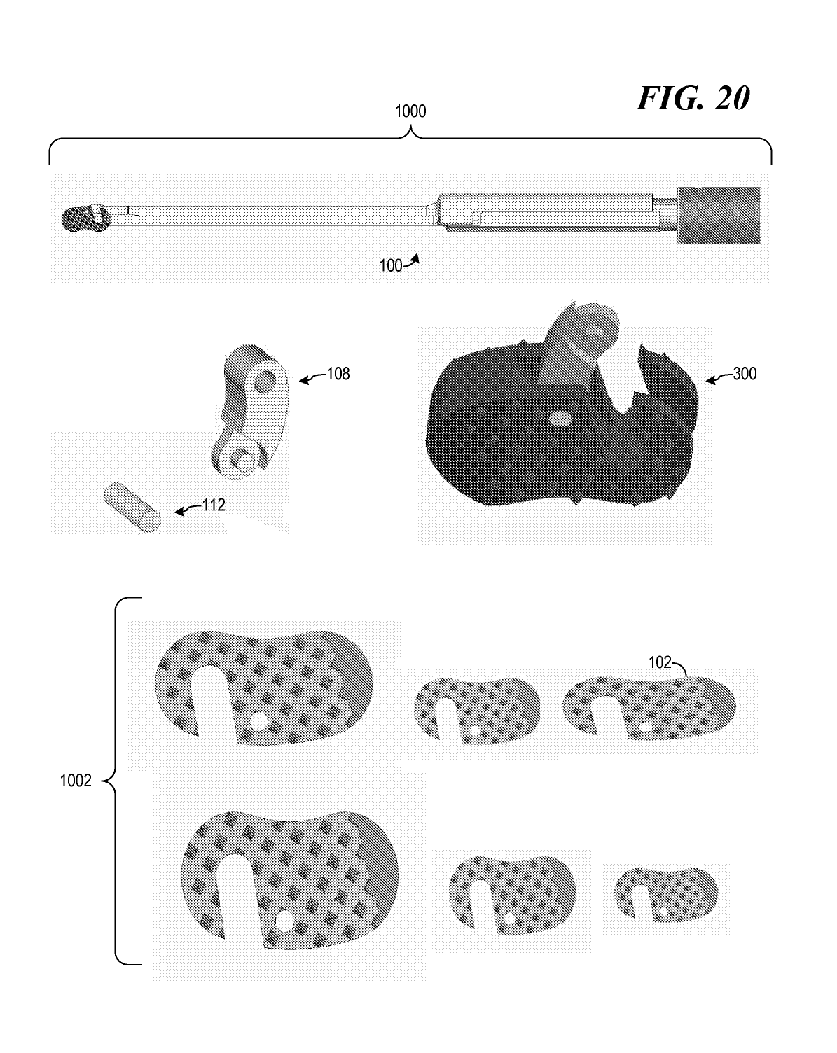

Referring now to FIG. 20, this drawing shows one possible embodiment of a rasp/trial member instrument kit 1000. The rasp/trial member instrument kit 1000 may include any of the steerable rasp/trial instrument embodiments described above such as instruments 100, 400 or 700. For illustrative purposes only, the instrument 100 is shown. In certain embodiments the instrument kit may include a plurality of rasp/trial members 1002 of various sizes and geometries. The rasp/trial members, such as the rasp/trial member 102 as shown, may include either rasp inserts, trial inserts or both. The surfaces of the plurality of rasp/trial members 1002 may vary in degrees of roughness or bone scraping ability. For example, if one of the plurality of rasp/trial members 1002 is to be used primarily as a trial member, then the rasp/trial member 102 may have a less textured surface then a rasp/trial member 102 that may be used to aggressively remove bone and produce hemorrhaging. Other rasp/trial members may also be used interchangeably such as rasp/trial members 702 and 402. The plurality of rasp/trial members 1002 may be provided as a rasp/trial link assembly. For example the rasp/trial member insert assembly 300 is shown, but rasp/trial member insert assembly 500 or rasp/trial member insert assembly 800 may also be used. Although only one rasp/trial member insert assembly 300 is shown in this figure a plurality may be provided in the kit 1000. The plurality of rasp/trial members 1002 may also be provided with the link member 108 and the pin 112 in order to allow assembly of the selected rasp/trial member 102 into a rasp/trial member insert assembly 300 for coupling to the steerable rasp/trial instrument 100. In other embodiments other link members such as 408 and 708 and the translation member 711 may be used as well as pins 412 and 712 to assemble and couple a rasp/trial insert assembly 500 or 800 to the steerable rasp/trial instrument 400 or 700.

Other embodiments for a surgical instrument may include:

1. A surgical instrument for posterior or lateral placement of a rasp between adjacent vertebrae comprising a first vertebrae and a second vertebrae, the instrument comprising:

a first member coupled to the rasp at a distal end of the first member and configured to facilitate rotation of the rasp relative to the first member;

a second member coupled to the first member and configured to translate relative to the first member;

an actuator coupled to the first and second member and configured such that rotation of the actuator about the first member and the second member translates one of the first member and the second member relative to the other; and

a connector member coupled to the rasp and a distal end of the second member and configured to pivot relative to the rasp and the second member such that movement of the second member relative to the first member rotates the rasp.

2. The instrument of embodiment 1 further comprising a pin securing the connector member to the rasp.

3. The instrument of embodiment 1 wherein the rasp further comprises a first cutting surface and a second cutting surface opposite to the first cutting surface.

4. The instrument of embodiment 3 wherein a distance between the first cutting surface and the second cutting surface decreases toward a distal end of the rasp.

5. The instrument of embodiment 1 further comprising a substantially U-shaped recess contained in the rasp and configured to accommodate the distal end of the first member.

6. The instrument of embodiment 5 further comprising a substantially U-shaped recess at the distal end of the second member and configured to accommodate a proximal end of the connector member.

7. The instrument of embodiment 1 further comprising a substantially U-shaped recess at the distal end of the second member and configured to accommodate a proximal end of the connector member.

8. The instrument of embodiment 1 further comprising a locking member configured to substantially fix a location of the first member relative to the second member through actuation of the locking member, thereby substantially fixing an orientation of the rasp relative to the first member.

Still other embodiments for a surgical instrument may include:

1. A surgical instrument for posterior or lateral placement of a rasp between adjacent vertebrae comprising a first vertebrae and a second vertebrae, the instrument comprising:

a first member coupled to the rasp at a distal end of the first member and configured to facilitate rotation of the rasp relative to the first member;

a second member coupled to the first member and configured to translate relative to the first member;

an actuator comprising a threaded section configured to couple the actuator to one of the first member and the second member, and the actuator configured such that rotating movement of the actuator about the first and second members translates one of the first and second members relative to the other; and

a connector member coupled to the rasp and a proximal end of the second member and configured to pivot relative to the rasp and the second member such that movement of the second member relative to the first member rotates the rasp.

2. The instrument of embodiment 1 further comprising a substantially U-shaped recess contained in the rasp and configured to accommodate the distal end of the first member.

3. The instrument of embodiment 2 further comprising a substantially U-shaped recess at the distal end of the second member and configured to accommodate a proximal end of the connector member.

4. The instrument of embodiment 1 further comprising a substantially U-shaped recess at the distal end of the second member and configured to accommodate a proximal end of the connector member.

5. The instrument of embodiment 1 further comprising a locking member configured to substantially fix a location of the first member relative to the second member through actuation of the locking member, thereby substantially fixing an orientation of the rasp relative to the first member.

Other embodiments for a surgical instrument may include:

1. A surgical instrument for posterior or lateral placement of a rasp between adjacent vertebrae comprising a first vertebrae and a second vertebrae, the instrument comprising:

a first member coupled to the rasp at a distal end of the first member and configured to facilitate rotation of the rasp relative to the first member;

a second member coupled to the first member and configured to translate relative to the first member;

an actuator comprising a threaded section configured to couple the actuator to one of the first member and the second member, and the actuator configured such that a rotating movement of the actuator translates one of the first member and second member relative to the other; and

a connector member coupled to the rasp and a distal end of the second member and configured to pivot relative to the rasp such that movement of the second member relative to the first member rotates the rasp.

2. The instrument of embodiment 1 wherein the connector member comprises a plurality of links coupled together for pivoting relative to each other.

3. The instrument of embodiment 1 further comprising a connector protrusion and a connector recess configured to couple the connector member to the first member such that the connector member is movable relative to the first member.

4. The instrument of embodiment 2 wherein the plurality of links comprises a first link and a second link.

5. The instrument of embodiment 4 further comprising a second connector protrusion and a second connector recess configured to couple the connector member for movement with the second member.

6. The instrument of embodiment 1 further comprising a locking member configured to substantially fix a location of the first member relative to the second member through actuation of the locking member, thereby substantially fixing an orientation of the rasp relative to the first member.

7. The instrument of embodiment 1 wherein the rasp further comprises a first cutting surface and a second cutting surface opposite to the first cutting surface such that a distance between the first cutting surface and the second cutting surface decreases toward a distal end of the rasp.

Further embodiments for a surgical instrument may include:

1. A surgical instrument for spine surgery, comprising:

an elongated member having a proximal end portion and a distal end portion;

an articulation member slidingly coupled to the elongated member, wherein the articulation member has a distal end portion and a proximal end portion;

an insertion linkage having a distal end portion and a proximal end portion, wherein the proximal end portion of the insertion linkage is pivotedly coupled to the distal end portion of the articulation member;

a rasp member, detachably rotatedly coupled to the distal end portion of the elongated member and rotatedly coupled to the distal end portion of the insertion linkage, wherein the rasp member includes at least one surface configured for traumatizing tissue;

an actuating mechanism coupled to the proximal end portions of elongated member and articulation member configured to move the articulation member relative to the elongated member.

2. The surgical instrument of embodiment 1 wherein the distal end portion of the articulation member is detachably coupled to the proximal end portion of the articulation member and the elongated member.

3. The surgical instrument of embodiment 1 wherein the actuating mechanism includes a locking member coupled to the elongated member.

4. The surgical instrument of embodiment 1 wherein the actuating mechanism includes an articulation knob threadedly coupled to the elongated member and coupled to the articulation member such that rotation of the articulation knob causes longitudinal movement of the articulation member with respect to the elongated member.

5. The surgical instrument of embodiment 1 wherein the actuating mechanism includes an impact surface coupled to the actuating mechanism.

6. The surgical instrument of embodiment 5 wherein the impact surface comprises a width that is greater than the width of the elongated member or the width of the guide member.

7. The surgical instrument of embodiment 1 wherein a top surface and an opposite bottom surface of the rasp member further comprise surfaces configured for traumatizing tissues.

8. The surgical instrument of embodiment 1 wherein a top surface and an opposite bottom surface of a distal end portion of the rasp member are tapered toward each other.

9. The surgical instrument of embodiment 1 wherein the at least one surface for traumatizing tissue comprises a plurality of teeth.

Still further embodiments for a surgical instrument may include:

1. A surgical instrument for spine surgery, comprising:

an elongated member having a proximal end portion and a distal end portion;

an articulation member slidingly coupled to the elongated member, wherein the articulation member has a distal end portion and a proximal end portion;

an insertion linkage having a distal end portion and a proximal end portion, wherein the proximal end portion of the insertion linkage is pivotedly coupled to the distal end portion of the articulation member;

a removable insert, detachably rotatedly coupled to the distal end portion of the elongated member and rotatedly coupled to the distal end portion of the insertion linkage, the removable insert having a height between a top surface and an opposite bottom surface in a range between 4 mm and 20 mm;

an actuating mechanism coupled to the proximal end portions of elongated member and articulation member configured to move the articulation member relative to the elongated member.

2. The surgical instrument of embodiment 1, wherein at least one surface of the top and bottom surfaces of the removable insert is further configured to traumatize tissue.

3. The surgical instrument of embodiment 2, wherein the at least one surface of the removable insert configured to traumatize tissue further comprises a plurality of teeth.

4. The surgical instrument of embodiment 1, wherein each of the top and bottom surfaces of the removable insert is further configured to traumatize tissue.

5. The surgical instrument of embodiment 1, wherein the actuating member is slidably coupled to the articulation member and threadably coupled to the elongated member.

6. The surgical instrument of embodiment 1, wherein the actuating member further comprises an impaction surface provided on a proximal end portion of the actuating member.

7. The surgical instrument of embodiment 1, wherein the distal end portion of the insertion linkage is rotatedly coupled to the removable insert via a pin.

8. The surgical instrument of embodiment 1, wherein the top and bottom surfaces of a distal end portion of the removable insert are angled toward each other.

Other embodiments for a method may include:

1. A method of traumatizing a pair of adjacent vertebral endplates comprising:

providing a surgical instrument having a pivoting distal removable insert, a proximal handle portion, a body portion, and a linkage member, positioned between the distal insert and the proximal handle portion, the distal removable insert having a first angular position relative to the body and the distal removable insert having textured top and bottom surfaces;

placing a leading end of the distal removable insert in a first position between two adjacent vertebral endplates;

moving the distal removable insert to a second position between the adjacent vertebral endplates by impacting the proximal end portion of the surgical instrument;

pivoting the distal removable insert to a second angular position relative to the body portion by rotating the handle about the body portion;

locking the second angular position of the distal insert; and

moving the distal removable insert to a third position between the adjacent vertebral endplates by impacting the proximal end portion of the surgical instrument.

2. The method of embodiment 1 further comprising:

removing the distal removable insert from between the adjacent vertebral endplates;

detaching the distal removable insert from the surgical instrument;

replacing the distal removable insert with a second distal removable insert chosen from a kit having a plurality of distal removable inserts.

3. The method of embodiment 2 wherein the plurality of distal removable inserts have a height between the top and the bottom surfaces in a range of 4 mm to 20 mm.

4. The method of embodiment 2 further comprising:

placing a leading end of the second distal removable insert in a first position between tow adjacent vertebral endplates;

moving the second distal removable insert to a second position between the adjacent vertebral endplates by impacting the proximal end portions of the surgical instrument;

pivoting the second distal removable insert to a second angular position relative to the body by rotating the handle about the body;

locking the second angular position of the second distal insert; and

moving the second distal removable insert to a third position between the adjacent vertebral endplates by impacting the proximal end portion of the surgical instrument.

Other embodiments for a surgical kit may include:

1. A surgical kit for spine surgery, comprising:

a surgical instrument comprising;
an elongated member having a proximal end portion and a distal end portion;
an articulation member slidingly coupled to the elongated member, wherein the articulation member has a distal end portion and a proximal end portion;
an actuating mechanism coupled to the proximal end portions of elongated member and articulation member configured to move the articulation member relative to the elongated member;

at least one removable inserts, wherein each of the removable inserts comprises;
an insertion linkage having a distal end portion and a proximal end portion, wherein the proximal end portion of the insertion linkage is pivotedly coupled to the distal end portion of the articulation member; and
a removable insert body, configured to be detachably rotatedly coupled to the distal end portion of the elongated member and rotatedly coupled to the distal end portion of the insertion linkage, the removable insert body having a height between a top surface and an opposite bottom surface in a range between 4 mm and 20 mm.

2. The surgical kit of embodiment 1, wherein the at least one removable insert comprises a plurality of removable inserts.

3. The surgical kit of embodiment 1, wherein the at least one of the top and bottom surfaces of the removable insert further comprises at least a portion of a surface configured to traumatize a vertebral endplate.

4. The surgical kit of embodiment 1, wherein the at least a portion of a surface comprises a plurality of teeth.

5. The surgical kit of embodiment 1, wherein the at least one removable insert is pivotally coupled to the insertion linkage via a pin.

The foregoing details provided regarding the embodiments of the invention have been presented primarily for the purposes of illustration and description. The details and drawings are not intended to be exhaustive listing of potential embodiments, nor should they limit the invention to the precise forms disclosed. Many modifications, combinations, and variations are possible in light of the above teachings while still remaining within the subject matter of the invention. It is intended that the scope of the invention is only limited by the Claims appended hereto.

1. A surgical instrument for spine surgery, comprising:
an elongated member having a proximal end portion and a distal end portion;
an articulation member slidingly coupled to the elongated member, wherein the articulation member has a distal end portion and a proximal end portion;
a link member having a distal end portion and a proximal end portion, wherein the proximal end portion of the link member is pivotably coupled to the distal end portion of the articulation member;
a rasp member, pivotably coupled to the distal end portion of the elongated member and pivotably coupled to the distal end portion of the link member wherein the rasp member includes at least one surface for traumatizing tissue; and
an actuating mechanism coupled to the proximal end portions of elongated member and articulation member configured to move the articulation member relative to the elongated member.

2. The surgical instrument of claim 1 wherein the proximal end portion of the link member is detachably coupled to the distal end portion of the articulation member and the rasp is detachably coupled to the elongated member.

3. The surgical instrument of claim 1 wherein the actuating mechanism includes a locking member temporarily positioned within the articulation member.

4. The surgical instrument of claim 1 wherein the actuating mechanism includes an articulation knob threadedly coupled to the elongated member and coupled to the articulation member such that rotation of the articulation knob causes longitudinal movement of the articulation member with respect to the elongated member.

5. The surgical instrument of claim 1 wherein the actuating mechanism includes an impaction knob coupled to the actuating mechanism.

6. The surgical instrument of claim 5 wherein the impact knob had an impaction area that is greater than a cross sectional area of the elongated member.

7. The surgical instrument of claim 1 wherein a top surface and an opposite bottom surface of a distal end portion of the rasp member are tapered toward each other.

8. The surgical instrument of claim 1 wherein the at least one surface for traumatizing tissue comprises a plurality of teeth.

9. The surgical instrument of claim 1 wherein the link member extends along a curved longitudinal axis.

10. A surgical instrument for spine surgery, comprising:
an elongated member having a proximal end portion and a distal end portion;
an articulation member slidingly coupled to the elongated member, wherein the articulation member has a distal end portion and a proximal end portion;
an insertion linkage having a distal end portion and a proximal end portion, wherein the proximal end portion of the insertion linkage is pivotedly coupled to the distal end portion of the articulation member;
a removable insert, detachably pivotably coupled to the distal end portion of the elongated member and pivotably coupled to the distal end portion of the insertion linkage, the removable insert having a height between a top surface and an opposite bottom surface in a range between 4 mm and 20 mm; and
an actuating mechanism coupled to the proximal end portions of elongated member and articulation member configured to move the articulation member relative to the elongated member.

11. The surgical instrument of claim 10, wherein at least one surface of the top and bottom surfaces of the removable insert is further configured to traumatize tissue.

12. The surgical instrument of claim 11, wherein at least one surface of the removable insert is configured to traumatize tissue further comprises a plurality of teeth.

13. The surgical instrument of claim 10 wherein at least one surface of the removable insert is substantially smooth.

14. The surgical instrument of claim 10, wherein the actuating mechanism is slidably coupled to the articulation member and threadably coupled to the elongated member.

15. The surgical instrument of claim 10, wherein the actuating member further comprises an impaction surface provided on a proximal end portion of the actuating member.

16. The surgical instrument of claim 10, wherein the distal end portion of the insertion linkage is rotatedly coupled to the removable insert via a pin.

17. The surgical instrument of claim 10, wherein the top and bottom surfaces of a distal end portion of the removable insert are angled toward each other.

18. A surgical instrument for spine surgery, comprising:
A generally cylindrical body having a distal end portion and a proximal end portion;
an articulation member slidingly coupled to the body and having a proximal end portion and a distal end portion having a recess;
an elongated member having a proximal end portion coupled to the body and a distal end portion;
a rasp member, pivotably coupled to the distal end portion of the elongated member and pivotably coupled to the distal end portion or the articulation member;
an actuating mechanism coupled to proximal end portion of the body,
a lock mechanism having a proximal end portion and a distal end portion, the lock mechanism having a first position wherein the distal end portion of the lock mechanism is positioned within the recess of the articulation member, such that the articulation member can not translate distally relative to the body, the lock mechanism further including a second position wherein the distal end portion of the lock mechanism is not positioned within the recess of the articulation member, such that the articulation member may translate in relation to the main body.

19. The surgical instrument of claim 18 wherein the locking mechanism is biased in the second position.

20. The surgical instrument of claim 18 further comprising an impaction knob coupled to the proximal end portion of the body.

21. The surgical instrument of claim 18 wherein the actuating mechanism includes an articulation knob threadedly coupled to the articulation member such that rotation of the articulation knob causes translation of the articulation member with respect to the body.

22. The surgical instrument of claim 18 wherein a top surface and an opposite bottom surface of a distal end portion of the rasp member are tapered toward each other.

23. The surgical instrument of claim 18 wherein at least one of the top and the bottom surfaces of the rasp member include a plurality of teeth.
